(19) United States
(12) Patent Application Publication
(10) Pub. No.: US 2023/0414941 A1
(43) Pub. Date: Dec. 28, 2023

(54) TREATMENT TEMPLATES

(71) Applicant: Pulse Biosciences, Inc., Hayward, CA (US)

(72) Inventors: Michael A. SMITH, Hayward, CA (US); Sukumar HIRANI, Hayward, CA (US); David J. DANITZ, San Jose, CA (US); Kevin L. MOSS, Lathrop, CA (US); Anita L. CROMPTON, Hayward, CA (US)

(21) Appl. No.: 18/336,907

(22) Filed: Jun. 16, 2023

Related U.S. Application Data
(60) Provisional application No. 63/354,997, filed on Jun. 23, 2022.

(51) Int. Cl. A61N 1/36

(52) U.S. Cl. CPC A61N 1/36014; A61N 1/328

(57) ABSTRACT

Described here are methods and apparatuses, including templates and template systems, for accurately positioning electrode applicator tips in order to treat larger predefined patterns of tissue. Also described here are methods and apparatuses for positioning, e.g., orienting and/or spacing, applicator tip(s) against the tissue while applying energy through some or all of the electrodes in the applicator tip.

### CLAIM OF PRIORITY

This patent application claims priority to U.S. Provisional Patent Application No. 63/354,997, titled “TREATMENT TEMPLATES,” filed on Jun. 23, 2022, which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Electric energy may be used in treatment of human cells and tissue including tumor cells, such as basal cell carcinoma, squamous cell carcinoma, and melanoma. For example, ultra-short, high-field strength electric pulses have been described for electroperturbation of biological cells. To treat tissues, including cancers, applicator tips (“tips” or “treatment tips”) may be applied to the tissue. Each applicator tip may include multiple electrodes that may be arranged, in some examples as an array, in a pattern that will influence the pattern of the applied energy and, therefore, the pattern of the effect of the application of energy to the tissue being treated. A variety of different applicator tips have been described, and these applicator tips may have different sizes and different arrangement of electrodes. For example, FIGS. 1A and 1B illustrate an example of a treatment tip that includes a plurality of electrodes, shown in this example as needle electrodes, that are arranged in two lines of five electrodes. In use, the final pattern of energy applied by the apparatus may be determined by shape of the tip as well as the arrangement of the tip, and in some cases the subset of electrodes of the tip to which energy is applied. This may be sufficient when treating small lesions, where the applicator tip may cover a surface area that includes all or essentially all of the lesion to be treated. However, when the surface area of the tissue and/or lesion(s) to be treated is greater than the effective area of the applicator tip it may be difficult to accurately position the applicator tips relative to the target tissue region(s) while still maintaining a good skin contact and a depth of treatment. It is also difficult to increase the size of the treatment area of the applicator tip that may still maintain good skin contact and depth of treatment.

For example, when treating large lesions, such as basal cell carcinoma (BCC), it may be useful to reposition a treatment tip of the apparatus one or more times over a large region of the tissue in order to sufficiently treat the target tissue area. What is needed are tools and methods for assisting a user (e.g., physician, doctor, nurse, medical practitioner, etc.) in placing and maintaining the applicator tip in a predictable and accurate manner.

### SUMMARY OF THE DISCLOSURE

Described herein are methods and apparatuses (e.g., devices and systems, including templates and template systems) for accurately positioning electrode applicator tips against a tissue to be treated by the applicator tip. Also, described herein are methods and apparatuses for positioning, e.g., orienting and/or spacing, applicator tip(s) against the tissue while applying energy through some or all of the electrodes in the applicator tip. The methods and apparatuses described herein may be used for treating a tissue and/or positioning relative to the tissue (e.g., a lesion, such as a tumor, cyst, growth, etc.). Additionally or alternatively, any of the apparatuses and methods described herein may be used for cosmetic indications, such as (but not limited to) treating scars, rosacea/rosacea-like dermatitis, wrinkles, oily skin, hyperhidrosis (e.g., excessive sweating), or the like. Such cosmetic indications typically have no symptoms other than the visible effects being treated and, while annoying and may lead to negative psychosocial consequences, are generally without medical consequences. For example, a sebaceous hyperplasia (SH) is one example of a cosmetic indication. SH treatment is considered an aesthetic/cosmetic treatment as SH is generally painless and generally without medical implications.

The template apparatuses described herein may be used to guide and/or secure the applicator tip(s) through a sequence of positions and/or orientations in a predetermined arrangement in order to treat a region of tissue that may be larger than the area of the applicator tip alone. The template apparatus may include multiple template guides, that overlap and/or may interlock with each other. Alternatively or additionally, each of the template guides in a series of template guides may engage and/or interlock with a frame (“base frame”) that couples or is coupled to the patient's tissue. In some examples the frame may be one of the template guides of the series (or a kit) of template guides, and subsequent template guides may engage with the frame and/or with other template guides. Each template guide of the template apparatus may be configured to provide one or more (e.g., a plurality) of applicator tip engagement positions (also referred to herein as seating positions) that engage with the one or more applicator tips and position the applicator tip in a predetermined manner, which may include a predetermined rotational orientation; once in position at each of the engagement positions, the applicator tip may contact the tissue at a predetermined location and (in some examples) orientation, and energy may be applied from the applicator tip to the tissue. For each template guide, an applicator tip may be sequentially moved to each of the engagement positions in the template guide so that the applicator tip contacts the tissue from each engagement position and energy may again be applied. After applying energy from all (or a subset of all, as may be applicable) of the engagement positions of a particular template guide, an additional template guide may be applied over or in place of the previous template guide. This process may be repeated until the entire treatment area is treated.

All or some of the template guides may include rotational orientation indicator(s) (e.g., marker, groove, etc.) indicating the rotational orientation of the applicator tip for all or some of the applicator positions when the applicator tip is positioned by the template guide. For example, the rotational orientation indicator(s) may include rotational orientation indicator(s) that align with and/or engage corresponding and/or complementary marking(s) and/or indicator(s) on the applicator tip. The rotational orientation indicator(s) may be markings and/or keyed members (e.g., tabs, projections, etc.). The complementary indicator(s) on the applicator tip may be complementary marking(s) and/or keyed engagement members that align with and/or engage the rotational orientation indicator(s) on the template guide(s). The proper alignment when repositioning an applicator tip multiple times to cover the treatment area may optimize skin healing and may avoid railroad track lines.

The template apparatuses described herein, including a plurality of template guides, may be configured to streamline and/or simplify the application of a plurality of treatments over a treatment area that is larger than the effective electrode contact region of the applicator tip. Moreover, the kits of the template guides according to the present disclosure allow a user to maintain a depth of treatment and a good skin contact through a large treatment area. For example, the template apparatuses may be configured so that a sequence of applicator positions and/or orientations may be applied to the treatment tissue in a series of sequential steps. For example, a template apparatus may include a plurality of template guides; each template guide may be configured to include one or a plurality of engagement positions that the user may use to optimize positioning of the applicator tip. In some examples all or some of the engagement positions may include rotational orientation indicator(s) to orient the applicator tip in both a gross planar position (e.g., in the plane of the tissue) and/or a rotational position. In some examples the template apparatus may be configured so that each of the template guides of the template apparatus indicates a single rotational orientation, so that the applicator tip(s) do not have to be rotated when applying energy from the one or more applicator position(s) on each template guide.

A user may secure the template apparatus over and/or against the tissue to be treated. In some examples the entire template apparatus may be secured to the tissue to be treated. In some examples just the base frame of the template apparatus is secured to the tissue to be treated. The one or more template guides may engage with the base frame (which may itself act as a template guide) in a predetermined orientation. The template apparatus (e.g. base frame) may be secured to the tissue in any appropriate manner, including adhesively. In some examples each of the template guides may be coupled to the base frame in a predetermined order (or in some examples, in any order) and the applicator tip(s) may be positioned by sequentially engaging each of the engagement positions on the template guide, contacting the tissue with the electrodes (e.g., in some examples deploying the electrodes) and applying energy (e.g., sub-microsecond pulses). The applicator tips may be rotationally oriented as indicated or guided by one or more rotational orientation indicators on all or some of the engagement positions of the template guide.

After an applicator tip has applied energy to the tissue from each of the one or more engagement positions of the template guide the next template guide may be applied (e.g., by inserting into the base frame) and the process may be repeated. In some examples the prior template guides may be removed (except for the base frame template guide) and replaced with the next template guide to be used, which may be organized in a sequential order. Alternatively in some examples the template guides may be sequentially removed to expose the subsequent template guides, or they may be added onto the prior template guides.

The template apparatuses described herein may therefore be used to apply consistent and complete treatment of the target tissue, even when the target tissue region is large (e.g., larger than the applicator tip area). The template apparatus may allow treatment with little or no treatment gaps and/or with minimal overlap between adjacent treatment applications. The use of a template apparatus with an applicator tip as described herein may provide numerous benefits as compared with the use of as single template, which may not permit close and controlled application or may limit the number of engagement positions, or with the use of guides such as tattoo or skin marking, which may be imprecise.

For example, described herein are template apparatuses for guiding the application of electrical energy to a tissue. Any of these apparatuses may include: a base frame; and a plurality of template guides configured to removably fit into the base frame, wherein each template guide of the plurality of template guides includes one or more applicator tip engagement position openings configured to receive a distal region of an applicator tip, further wherein at least some of the applicator tip engagement position openings of the template guides may be combined to form an enlarged treatment pattern. The enlarged treatment pattern covers a larger area than any of the individual applicator tip engagement position openings.

For example, a template apparatus for guiding a position of an applicator tip (or guiding an application of electrical energy to a tissue) may include: a base frame; and a plurality of template guides configured to removably fit into the base frame, wherein each template guide of the plurality of template guides includes one or more applicator tip engagement position openings configured to receive a distal region of an applicator tip, and further wherein the one or more applicator tip engagement position openings of the plurality of template guides are configured to form an enlarged treatment pattern after at least some of template guides of the plurality of template guides are sequentially held within the base frame in one or more orientations so that an applicator tip may be received by the one or more applicator tip engagement position openings of the at least some of the template guides of the plurality of template guides.

For example, a template apparatus for guiding an application of electrical energy to a tissue may include: a base frame; and one or more template guides configured to removably fit into the base frame in one or more orientations, wherein each template guide of the one or more template guides includes one or more applicator tip engagement position openings configured to receive a distal region of an applicator tip, and further wherein the one or more applicator tip engagement position openings of the one or more template guides are configured to form an enlarged treatment pattern after at least one of the one or more template guides held within base frame in more than one orientation and/or more than one of the one or more template guides are sequentially held within the base frame so that an applicator tip may be received by the one or more applicator tip engagement position openings of the one or more template guides.

In some examples, a template apparatus for guiding a position of an applicator tip is provided. The template apparatus may comprise: a base frame; and a plurality of template guides configured to removably fit into the base frame in one or more orientations, wherein each template guide of the plurality of template guides includes one or more applicator tip engagement position openings configured to receive a distal region of an applicator tip, and further wherein the one or more applicator tip engagement position openings of a first template guide of the plurality of template guides forms an enlarged treatment pattern with: 1) the one or more applicator tip engagement position openings of a second template guide of the plurality of template guides, or 2) the one or more applicator tip engagement position openings of the first template guide in a second orientation of the one or more orientations.

In any of these apparatuses, the base frame may include an adhesive configured to secure the base frame to a patient. The adhesive may be on a back surface of the base frame, including on a portion of the back or on the entire back surface of the frame, including on a protruding tab (e.g., attachment projections) or another region. The adhesive may be a biocompatible adhesive (e.g., a skin-compatible adhesive). For example, the apparatus may include attachment projections extending from the base frame that are configured to attach the base frame to the tissue. The frame may be provided with adhesive already applied (e.g., adhesive tape, etc.) or the adhesive may be applied by the user. In some implementations, instead of adhesive or in addition to the adhesive, a user my apply an external tape.

Any of these apparatuses may be configured to align the rotational position of the applicator relative to the template(s). For example, at least some of applicator tip engagement position openings may include one or more rotational orientation indicators for rotationally aligning the applicator tip within the applicator tip engagement position openings. The one or more rotational orientation indicators may include markings (e.g., lines, dots, letters, numbers, colors, etc.) that may be configured to match markings on the applicator tip. In some examples the one or more rotational orientation indicators include engagements that engage with a region of the applicator tip. For example, the one or more rotational orientation indicators may include projections (tabs, teeth, etc.) that are configured to engage with the applicator tip in a particular orientation.

Any of these apparatuses may be configured so that each template guide engages with the base frame so that the template guide is held in a predetermined alignment relative to the base frame (and therefore relative to other template guides in the series of template guides that are part of the template apparatus). Thus, the template guides may be indexed relative to the base frame. For example, these apparatuses may include an indexing member on the base frame and configured to engage each template guides of the plurality of template guides and to releasably orient the template guide in the base frame. The indexing member may comprise a tab configured to engage with a tab receiver on each template guides of the plurality of template guides. The same template guide may be uniquely indexed to the base frame, so that it may only engage with the base frame in a single orientation, or a template guide may be configured to index to the base frame in a plurality of different orientations. For example, a template guide may be configured to engage with the base frame in configurations that are rotated by a predetermined amount (e.g., 90 degrees, 180 degrees, etc.).

The index may be a tab or projection. In some cases the template guide may also be indexed in part by fitting into the template window of the base frame in a predetermined orientation. The template guide(s) may be configured to fit snuggly into the template window of the base frame (e.g., with a tolerance of 1 mm or less, e.g., 0.8 mm or less, 0.7 mm or less, 0.6 mm or less, 0.5 mm or less, 0.4 mm or less, 0.3 mm or less, etc.). In some examples, the base frame may include a lock that engages the template guide to secure it within the template window of the base frame. The lock may bias (e.g., push) the template guide in the base frame in a predetermined manner to secure it into position. For example, the apparatus may include a spring-driven arm or tab that holds the template guide within the template window of the base frame.

The template guides may be separate from the base frame and may be removable and insertable into the base frame (e.g., into a template window of the base frame), and/or the template guides may be movably coupled to the base frame, so that they may operate as a unit. For example, the plurality of template guides may be hinged to the base frame. In some examples the plurality of template guides is hinged to the base frame by one or more of: a dowel pin, a c-shaped, a ball joint, and a living hinge.

Any of these apparatuses may include an engagement member on each base frame configured to engage with each template guide to removably secure the template guide in the base frame and against a tissue. The engagement member may be part of the index member, or it may be separate (e.g., may hold a template guide in position regardless of the orientation of the template guide relative to the base frame. In some examples the base frame may include an engagement member such as a pocket, a tab, or a latch. The engagement member may secure (or may help secure) the template guide in the template window of the base frame.

In any of these examples the base frame may be flexible and configured to conform to a curved tissue surface (e.g., a curved tissue surface). The template guide may be flexible and configured to conform to the tissue surface. For example, the base frame and/or template guide may be formed of a polymeric material such as a silicone, ethylene vinyl acetate (EVA), polyethylene (PE), thermoplastic elastomer (TPE), etc. of softer durometers. In any of the apparatuses described herein the base frame and/or templates may be formed of a relatively soft (e.g., compliant) durometer material, such as a material having a durometer, for example, of between about 15 A and 60 A on the Shore A scale (e.g., 25 A-50 A, 35 A and 45 A, etc.).

The template apparatuses described herein, including the base frame and a plurality of template guides that engage with the base frame by, e.g., fitting into a template window of the base frame, may generally be configured so that they form a pattern of electrical energy on and/or within the target tissue when used to position and orient a treatment tip. Each template guide may include a plurality of applicator tip engagement position openings that guide and/or hold an applicator tip so that a pattern in the tissue formed by multiple passes with the same (or different) applicator tips may be reliably and quickly (highly accurately) produced. The energy applied by the applicator tip, via the electrodes, may spread out and may penetrate the tissue (e.g., in some examples up to about 5 mm deep), including adjacent regions. The enlarged treatment pattern may cover a much larger surface area with electrical therapy or treatment than possible without the template apparatus. In general each applicator tip typically includes an array of two or more (e.g., 5, 8, 9, 15, etc.) electrodes arranged in an applicator tip pattern. The applicator tip pattern may be, for example, 3×3, 5×5, 7×7, 10×10, etc. and may include two or three or more rows of electrodes. The array of electrodes may be arranged, for example, in a square for convenience, though other configurations are possible. The electrode may be any appropriate type of electrode, including (but not limited to) needle electrodes, flat (e.g., surface) electrodes, plate electrodes, etc. The enlarged treatment pattern formed by the template apparatuses described herein may generally be much larger than the electrode pattern of a single applicator tip.

The electrode pattern of an electrode tip may be smaller and/or may be surrounded by a region without electrodes on the distal tip region of the applicator tip, which may make it more difficult (in the absence of a template apparatus as described here) to align and apply adjacent treatments. In any of these apparatuses, the applicator tip engagement position openings that guide and/or hold the distal end of the applicator tip may be configured to provide continuous and non-overlapping coverage of the tissue.

In any of these examples, at least some of the template guides are configured to removably fit into the base frame in two or more rotational orientations. For example, in some implementations at least some of the template guides may be configured to be secured in a first configuration relative to the base frame and may also be configured to be secured to the base frame in a second configuration that is rotated (e.g., 90 degrees, 180 degrees, 270 degrees, etc.) relative to the first configuration. Thus, the first template guide may be configured to engage with the base frame in an orientation that is 90 degrees offset from the second template guide.

As mentioned, any of these template apparatuses may be configured to both position and orient the applicator tip (e.g., electrode array) relative to the tissue. For example, a template apparatus for guiding the application of electrical energy to a tissue may include: a base frame; a plurality of template guides configured to removably fit into or couple with the base frame in a predetermined orientation, wherein each template guide of the plurality of template guides includes one or more applicator tip engagement position openings configured to receive a distal region of an applicator tip and to hold the applicator tip in a fixed position and orientation relative to the tissue. Further, at least some of the applicator tip engagement position openings include a rotational orientation indicator configured to align with and/or engage a complementary indicator on the distal region of the applicator tip.

A template apparatus for guiding the application of electrical energy to a target tissue may include: a base frame configured to be adhesively secured to a patient; a plurality of template guides configured to removably fit into the base frame in one or more predetermined orientations, wherein each template guide of the plurality of template guides includes one or more applicator tip engagement position openings configured to hold a distal region of an applicator tip in a fixed position and orientation relative to the target tissue, further wherein at least some of the applicator tip engagement position openings include a rotational orientation indicator configured to align with and/or engage a complementary indicator on the distal region of the applicator tip.

Also described herein are methods of using any of these apparatuses. According to some examples, a method of applying electrical energy to a target tissue using an applicator tip may include: attaching a base frame of a template apparatus to a patient; and applying energy in a predetermined pattern over the target tissue by sequentially, for each (or a subset of) of a plurality of template guides: inserting a template guide of the plurality of template guides into the base frame, and applying energy to the target tissue from an applicator tip inserted into each of one or more applicator tip engagement position openings of the template guide.

For example, a method of using a template apparatus to guide positioning of an applicator tip may include: attaching a base frame of a template apparatus to a treatment area over a target tissue; inserting a first template guide into the base frame in a first orientation; applying energy to the target tissue from an applicator tip inserted into each of one or more applicator tip engagement position openings of the first template guide; inserting a second template guide into the base frame or re-inserting the first template guide into the base frame in a second configuration; and applying energy to the target tissue from the applicator tip inserted into each of one or more applicator tip engagement position openings of the second template guide or of the first template guide in the second orientation.

In any of these methods, attaching the base frame comprises adhesively securing the base frame to the patient. For example, attaching the base frame may comprise attaching a flexible base frame so that the base frame conforms against the patient.

The template guides may be inserted into the base frame in a predetermined (e.g., numerically ordered) sequence, or they may be inserted into the base frame and used in any desired sequence. The template guides may be engaged with the base frame, including by engaging with an indexing member so that they are oriented in a predetermined manner. The template guides may be secured (removably secured) within the base frame, e.g., within a template window of the base frame. Inserting the first template guide may comprise inserting the first template guide into the base frame so that an index member on the base frame engages with the first template guide. The template guide may be secured in the base frame by an engagement member. For example, inserting the first template guide may comprise securing the first template guide within the base frame using an engagement member on the base frame.

As mentioned above, the methods and apparatuses described herein may be used for guiding and positioning any appropriate applicator, such applicator may apply any appropriate energy. In particular, these methods and apparatuses may use an applicator to apply sub-microsecond pulses of electrical energy and may use an applicator appropriate for applying high-voltage, sub-microsecond electrical energy. For example, the methods and apparatuses described herein may be used with and/or may be part of a system or method including any of the following applicator tips and/or pulse generators, each of which is herein incorporated by reference in its entirety: U.S. Pat. No. 11,167,125, titled “TREATMENT TIP WITH PROTECTED ELECTRODES,” filed Jan. 14, 2019; U.S. Pat. No. 10,857,347, titled “TREATMENT INSTRUMENT AND HIGH-VOLTAGE CONNECTORS FOR ROBOTIC SURGICAL SYSTEM,” filed Mar. 13, 2018; U.S. application Ser. No. 16/980,347, titled “MOVING ELECTRODES FOR THE APPLICATION OF ELECTRICAL THERAPY WITHIN A TISSUE,” filed Mar. 11, 2019; U.S. application Ser. No. 16/789,350, titled “HIGH-VOLTAGE CATHETERS FOR SUB-MICROSECOND PULSING,” filed Feb. 12, 2020; PCT Application no. PCTUS2021035146, titled “HIGH-VOLTAGE MINIMALLY INVASIVE APPLICATOR DEVICES FOR SUB-MICROSECOND PULSING,” filed Jun. 1, 2021; U.S. application Ser. No. 16/940,381, titled “NANOSECOND PULSED ELECTRIC FIELD SYSTEM,” filed Jul. 27, 2020; and U.S. application Ser. No. 17/590,790, titled “UNIVERSAL HANDPIECE FOR ELECTRICAL TREATMENT APPLICATOR,” filed Feb. 1, 2022.

Thus, applying energy may comprise applying sub-microsecond pulsed energy, e.g., applying an electric field with a sub-microsecond pulse width of between about 0.1 nanoseconds (ns) and 1000 ns, or shorter, such as 999 ns to 1 picosecond; the energy may be applied at high voltage, such as (but not limited to) 5 kilovolts per centimeter (kV/cm) or more (e.g., 5 kV/cm to 500 kV/cm, 7 kV/cm or more, 10 kV/cm or more, etc.).

Applying energy may include sequentially holding a distal end region of the applicator tip into each of the applicator tip engagement position openings, contacting the target tissue with one or more electrodes of the applicator tip and applying energy to the one or more electrodes.

Any of these methods may include aligning the applicator tip to a rotational orientation indicator on the first template guide and the second template guide before applying energy to the target tissue.

Any number of template guides may be used (and/or reused in different orientations). For example, any of these methods may include inserting a third (or more) template guide(s) into the base frame and applying energy to the target tissue from the applicator tip inserted into each of one or more applicator tip engagement position openings of the template guide.

In any of these methods, the method may include removing the template guide from the base frame before inserting the next template guide into the base frame.

Inserting the template guide (e.g., the first template guide or subsequent template guides) into the base frame may include inserting the first template guide into a window of the base frame. For example, inserting the first template guide into the base frame may include rotating the first template guide from a side of the base frame to which the first template guide is attached. In any of these methods the base frame may also be configured as a template guide (e.g., including one or more applicator tip engagement position openings) and may be used to apply energy from the applicator tip first or last in the series of template guides.

Also described herein are methods of engaging a plurality of template guides with a base frame. These methods may be performed without placing the template guides onto the tissue (e.g., without treating a patient). For example, a method of engaging each of a plurality of template guides with a base frame, wherein each template guide of the plurality of template guides includes one or more applicator tip engagement position openings configured to hold an applicator tip, may include: sequentially inserting each (or at least some) of the template guides of the plurality of template guides into the base frame so that each template guide is held by the base frame and so that the applicator tip engagement position openings of each template guide do not coincide with applicator tip engagement position openings of any other template guide when held within the base frame; and removing each template guide from the base frame.

Sequentially inserting each of the template guides further may include reinserting one or more of the template guides into the base in a different orientation. The different orientation may include a rotation of the one or more of the template guides (e.g., between 90 and 180 degrees about the midpoint of the base window). In some implementations, the method of engaging a plurality of template guides with a base frame may include: inserting a first template guide of the plurality of template guides into the base frame so that the first template guide is held by the base frame with an applicator tip engagement position openings of the first template guide in first respective locations; removing the first template guide from the base frame; and inserting a second template guide of the plurality of template guides into the base frame such that respective locations of an applicator tip engagement position openings of the second template guide do not coincide with the first respective locations. In addition or instead of inserting the second template guide into the base frame, the method may comprise reinserting the first template guide into the base frame in a different orientation. Inserting a respective template guide may include engaging an index member on the base frame with the respective template guide. In some examples inserting a respective template guides may comprise securing the respective template guide within the base frame using an engagement member on the base frame. For example, inserting the template guide into the base frame may include inserting the template guide into a window of the base frame.

Also described herein are methods and apparatuses in which the template apparatus includes one or more grids. For example, describe herein are template apparatuses for guiding an application of electrical energy to a tissue, comprising: one or more grids, wherein each grid includes one or more mating engagement configured to engage with a complimentary mating engagement of an applicator tip to hold the applicator tip in a predetermined position and/or orientation relative to a tissue being treated. The one or more mating engagements may be configured to form an enlarged treatment pattern after applicator tip is engaged with each of the one or more mating engagements of the one or more grids. These apparatuses may include a base frame, and the one or more grids may be configured to removably fit into the base frame in one or more orientations.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIGS. 1A-1B illustrate an example of an applicator tip that may be used with any of the template apparatuses described herein.

FIGS. 2A-2D illustrate example patterns for treatment templates having different sized treatment tips.

FIGS. 3A and 3B illustrate enlarged treatment patterns for applying energy to tissue using markers on the tissue.

FIGS. 4A-4C illustrate one example of a template apparatus including a base frame (FIG. 4A) configured to hold a first template guide (FIG. 4B) and a second template guide (FIG. 4C).

FIGS. 5A-5B illustrate top views of a base frame holding an example of a first template guide (FIG. 5B) and an example of a second template guide (FIG. 5A).

FIGS. 6A and 6B illustrate the operation of a template apparatus such as the one shown in FIGS. 5A-5B.

FIGS. 7A-7F illustrate another example of a template apparatus. FIG. 7A shows a base frame. FIGS. 7B-7F illustrate a series of five template guides that may be inserted into the base frame of FIG. 7A.

FIGS. 8A-8J illustrate the use of a template apparatus such as the one shown in FIGS. 7A-7F. FIGS. 8A-8E illustrate the use of a first template guide with an applicator tip inserted into the engagement position. FIGS. 8A-8C show perspective, front and back views, respectively of the applicator tip engaged with the template guide. FIGS. 8D and 8E show bottom and top views, respectively, of the template guide (within a base frame) with the applicator tip of an applicator inserted into the engagement position. FIGS. 8F-8G show perspective, front and back views, respectively, of a second template guide (within the base frame) with the applicator tip engaged with the engagement position of the second template guide. FIGS. 8I and 8J show bottom and top views, respectively, of the second template guide within the base frame.

FIGS. 9A-9C illustrate the examples of base frames as described herein. FIG. 9A shows an example of a base frame with a tapered corner 907 and FIG. 9B shows an example of a base frame with a retaining pocket 944 in one corner. In various implementations, retaining pockets 944 may be placed in multiple corners, for example, in 2, 3, etc., or all corners of the base frame. FIG. 9C shows an example of the base frame with a first template guide inserted therein.

FIGS. 10A-10G show one example of a template apparatus as described herein. FIG. 10A shows an example of a base frame. FIGS. 10B-10F show examples of template guides (guides 1-5, respectively), each having progressively more engagement positions and orientations. FIG. 10G illustrates a perspective view of the base frame and all five template guides.

FIG. 11 shows an enlarged view of one example of an engagement portion of a base frame engaging with an applicator tip, also showing alignment of rotational orientation indicators (shown as fiducial markers in this example).

FIGS. 12A-12D illustrate an example of a template apparatus including a base frame (FIG. 12A) and three template guides (FIGS. 12B-12D, respectively) that may be serially (and in some cases, sequentially) used with the base frame. FIG. 12E illustrates a perspective view of the base frame and all three template guides.

FIGS. 13A-13E illustrate an example of a template apparatus including a base frame (FIG. 13A) and four template guides (FIGS. 13B-13E, respectively) that may be serially (and in some cases, sequentially) used with the base frame. FIG. 13F illustrates a perspective view of the base frame and all four template guides.

FIG. 14 is an example of a template guide (similar to that shown in FIG. 12B) that includes tolerance offsets within the engagement position and orientation opening.

FIGS. 15A-15C illustrate an example of a template apparatus. FIG. 15A schematically illustrates the target grid pattern. FIGS. 15B and 15C show first and second template guides, respectively, within a base frame that may be used to achieve the grid pattern of FIG. 15A using an applicator tip.

FIGS. 16A-16C illustrate examples of flexile template apparatuses that may be used on flat or curved tissue surfaces.

FIGS. 17A-17C illustrate another example of a template apparatus. FIG. 17A schematically illustrates the target grid pattern for use, for example, with a 7.5×7.5 mm applicator tip. FIGS. 17B and 17C show a template guide that may be used in two different positions within a base frame to achieve the grid pattern of FIG. 17A using the 7.5×7.5 mm applicator tip.

FIGS. 18A-18D illustrate another example of a template apparatus. FIG. 18A schematically illustrates the target grid pattern for use, for example, with a 5×5 mm applicator tip. FIG. 18B shows a first template guide used with a base frame and FIGS. 18C and 18D show a second template guide that may be used in two different positions within the base frame to achieve the grid pattern of FIG. 18A using the 5×5 mm applicator tip.

FIGS. 19A-19C illustrate yet another example of a template apparatus. FIG. 19A schematically illustrates the target grid pattern for use, for example, with both a 5×5 mm applicator tip and a 10×10 mm applicator tip. FIG. 19B shows a first template guide used with a base frame (which may be rotated 90 degrees to achieve a second pattern) and FIG. 19C shows a second template guide that may be used with the base frame to achieve the grid pattern of FIG. 19A using the 5×5 mm applicator tip.

FIGS. 20A-20E illustrate a further example of a template apparatus. FIG. 20A schematically illustrates the target grid pattern for use, for example, with a 5×5 mm applicator tip. FIGS. 20B-20E shows four template guides that may each be used with a base frame, some of which may be rotated 90 degrees, 180 degrees or 270 degrees to achieve a target pattern of FIG. 20A using the 5×5 mm applicator tip.

FIG. 21 illustrates an example of a template apparatus including template guides that are movably coupled with the base frame. In FIG. 21 the template guides are coupled to the base frame by a living hinge.

FIGS. 22A and 22B show examples of template apparatuses including template guides that are movably coupled with the base frame by a hinge pin or dowel.

FIG. 23 is an example of a template apparatus including a template guide that is movably coupled with the base frame by a C-bracket.

FIG. 24 is an example of a template apparatus including a template guide that is movably coupled with the base frame by a ball joint.

FIGS. 25A-25C illustrate another example of a template apparatus including a template guide that is movably coupled with the base frame by a C-bracket

FIGS. 26A-26E illustrate an example of a template apparatus including a template guide that is movably connected with the base frame by a ball joint (e.g., ball and socket).

FIG. 27 shows an example of a template apparatus including three template guides movably coupled to the base frame.

FIG. 28 shows another example of a template apparatus including a template guide movably coupled to the base frame.

FIGS. 29A-29E illustrate an example of a template apparatus including a target treatment pattern grid having mating engagements that engage with the distal end of the applicator tip. FIG. 29A shows an example of a grid and FIG. 29B-29C show an example of a distal end of an applicator tip engaged with the grid of FIG. 29A. FIGS. 29D-29E demonstrate a complimentary-shaped mating engagement on the distal end of the applicator tip corresponding to the mating engagement(s) on the grid.

FIGS. 30A-30J illustrate one method of template sizing as described herein.

### DETAILED DESCRIPTION

The methods and apparatuses (e.g., devices and systems) described herein include templates for guiding a user (e.g., doctor, surgeon, nurse, technician, etc.) in positioning an applicator tip and/or applying energy to a patient using an applicator tip, as well as the methods of using such apparatuses. These methods and apparatuses may be particularly helpful for treating larger lesions and may avoid problems when applying multiple adjacent treatments using an applicator tip. These methods and apparatuses may minimize gaps between electrode contact (and therefore treatment) regions, assist in maintaining a good skin contact and a depth of treatment, and may rotationally orient the treatment tips to more uniformly distribute the therapeutic energy. Thus, these methods and apparatuses may minimize errors in applying therapy, particularly where the patient's skin is irregular (e.g., wrinkled, stretched, supported by cellulose, etc.) and may generally ensure proper placement and orientation of the electrode applicator tip(s). In general, controlling the alignment when applying multiple applications may help with skin healing and may avoid undesirable lines (“railroad track lines”) in the skin.

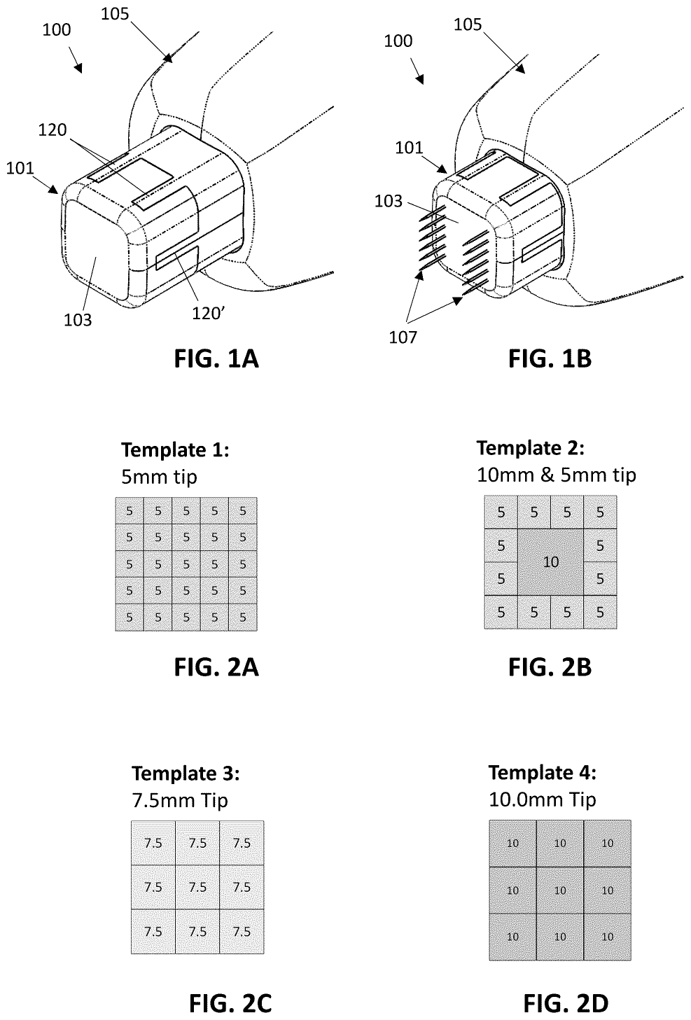

In general, the methods and apparatuses described herein may be used with any appropriate tips, including tips such as that shown in FIGS. 1A-1B. In this example the applicator tip 100 includes a proximal housing region 105 and a distal tip 101. The distal tip 101 may be driven back into the proximal housing 105, for example, by pushing against the distal face 103 of the distal tip 101, to expose the electrodes 107. In FIG. 1B the distal tip 101 is driven back into the housing 105 so that the array of electrodes 107 is exposed. This example shows an array of 2 rows of six needle electrodes.

As mentioned, any appropriate tip shape may be used. These tips may include more or fewer electrodes, and/or may include different types of electrodes, such as plate electrodes, wire electrodes, or surface (e.g., flat) electrodes. The applicator tip shown in FIGS. 1A-1B retracts, however in general, the methods and apparatuses described herein may be used with applicator tips that do not retract, or that extend outwards from the tip (rather than remain stationary while the tip retracts). The array of electrodes 107 shown in FIGS. 1A-1B is an approximately 5 mm×5 mm array (e.g., having 5 mm between the outermost needle electrodes), but any other size array (including non-square arrays) may be used. For example, arrays of 2.5 mm×2.5 mm, 7.5 mm×7.5 mm, 10 mm×10 mm, etc. may be used.

In the example applicator tip shown in FIGS. 1A-1B the distal tip region also includes a set of markings 120, 120′ on the sides of the distal tip 101 (also referred to as the tip housing). In this example, there are two markings 120 on the one (e.g., top) side indicating the side that is perpendicular to the line of electrodes, and one marking 120′ on the adjacent side, indicating the side that is parallel to the line of electrodes. As will be described in greater detail below, in some examples, the template apparatuses described herein may use these tip markings to rotationally orient the applicator tip. In some examples the tip housing may include one or more notches or protrusions on one or more sides that may also or alternatively be used for orientating the applicator tip.

It may be desirable to form a larger region of treatment than may be possible using a single tip (e.g., larger than a 25 mm² area treated by a single 5×5 applicator tip shown in FIGS. 1A-1B). For example, FIGS. 2A-2D illustrate enlarged treatment patterns that may be desired. In FIG. 2A the enlarged treatment pattern is a 25 mm×25 mm square and can be divided up into 25 different treatments using a 5 mm tip. In FIG. 2B the example enlarged treatment pattern is a 20 mm×20 mm square, shown divided up for treatment by both a 10 mm×10 mm tip and a 5 mm×5 mm applicator tip. FIGS. 2C and 2D show examples of 22.5 mm×22.5 mm and 30 mm×30 mm enlarged treatment patterns shown divided for treatment by 7.5 mm×7×5 mm applicator tips and 10 mm×10 mm tips, respectively. In general, such patterns may be helpful to cover a larger lesion, but may be difficult to achieve, as aligning the tips may be hard to do, even where markers are made on the subject's tissue (e.g., skin) to be treated. In addition, as shown in FIG. 1B, the electrodes may not be uniformly distributed across the treatment tip, thus it may be beneficial to rotationally alternate the orientation of the applicator tip while treating (e.g., rotating by 90 degrees between adjacent treatments), to approximate a more uniform treatment coverage and minimize any cosmetic damage due to needle penetration.

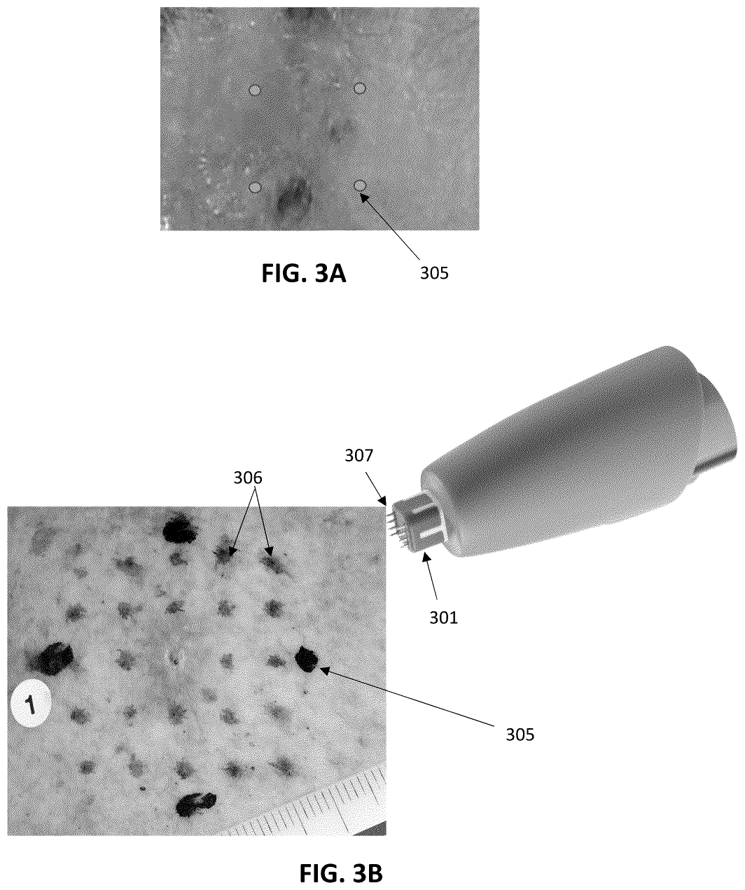

FIGS. 3A and 3B illustrate an example of one approach, in which a treatment region is manually marked 305 (e.g., by fiducial markings, such as dots or “tattoos”), such approach having certain shortcomings, as explained below. In this example, as shown in FIG. 3B, larger markings may be made at the boundary of the treatment region and the treatment region may be sub-divided up into a grid (and the grid marked by dots 306 at the corners) based on the size of the treatment tip (e.g., 5 mm×5 mm). To treat the patient, the corners of the grids (dots 306) may be aligned manually with the applicator tip 301 and treatment applied through the electrodes 307 using multiple cycles to cover the full lesion. In addition to requiring extra time for manually placing the dots or tattoos, in some examples this technique may also leave a margin around the treatment area, which may be undesirable. Alternatively the grid may be adjusted so that the margin is reduced or eliminated.

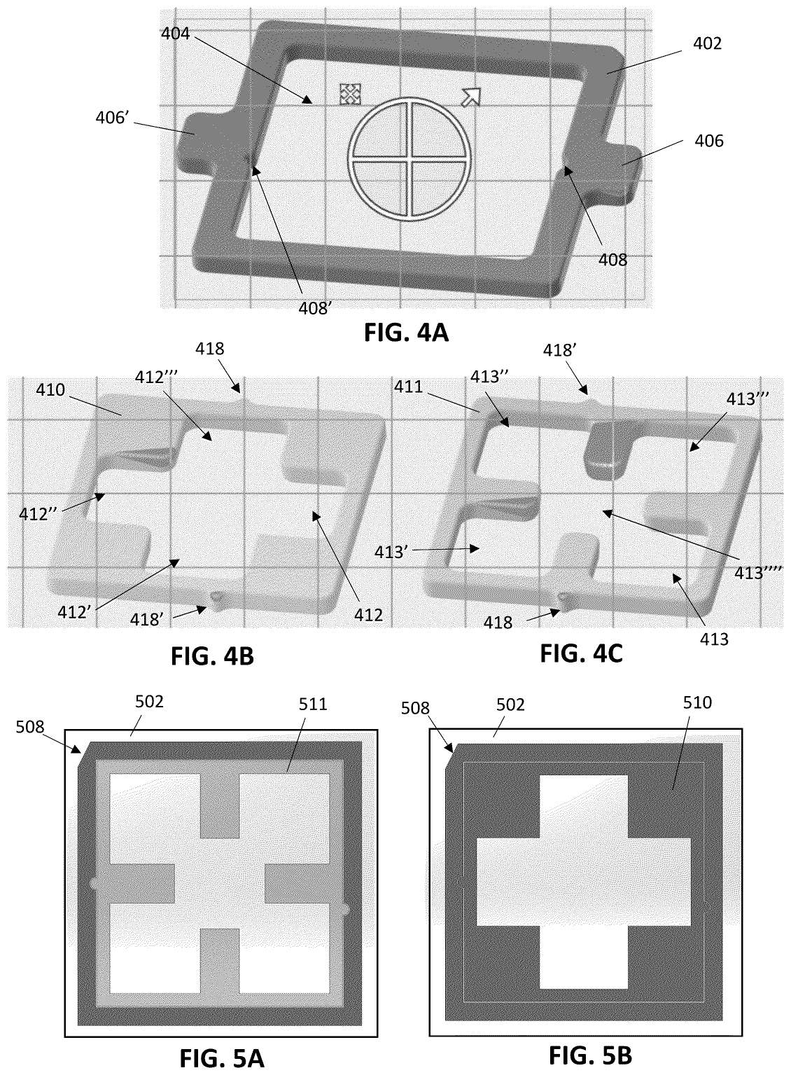

In contrast to the manual method described in FIGS. 3A and 3B, a template apparatus may instead be used to more rigorously control the application of energy to a target tissue in a more uniform manner. A template apparatus may include a base frame that may be secured to the patient over the target tissue, and one or more (e.g., two or more, three or more, etc.) template grids that may be sequentially held by the base frame and used to guide the applicator tip in applying energy to the tissue. For example FIG. 4A shows a first example of a base frame 402 of a template apparatus. In this example the base frame includes a template window 404 that is open and configured to hold each of the template guides (shown in FIGS. 4B and 4C) therein. The base frame 402 in this example includes a pair of index members 408, 408′ that are configured as notches or cut-out regions that may engage with a complimentary projection 418, 418′ on the template guide. In FIG. 4A the base frame also includes a pair of attachment projections 406, 406′ extending from the frame and configured to be used to interface with an adhesive (e.g., tape). The attachment projections may be wings, tabs, etc. The base frame may be secured to the patient by securing the attachment projection to the patient's body, using a bandage, an adhesive (e.g., a tape, glue, etc.). In some implementations instead of or in addition to the attachment projections, the tissue-facing surface of the base frame may be adhesive-backed to secure the templates to the treatment area.

FIGS. 4B and 4C illustrate examples of first 410 and second 411 template guides that may fit into (and may be removably secured within) the base frame 402. For example FIG. 4B shows a first template guide that includes a pair of complimentary projections 418, 418′ that may index the template guide with the base frame. The template guides may be sequentially inserted into the template window 404 of the base frame 402, and a user may insert a distal end of an applicator tip into any of the applicator tip engagement position openings 412, 412′, 412″, 412″. Once inserted, the applicator tip may be deployed (e.g., contacting the tissue to be treated with the electrode(s)), to deliver the electrical energy to the tissue. Thereafter, the electrodes may be removed from the tissue and/or the applicator tip may be removed from the applicator tip engagement position opening 412 and moved to the next applicator tip engagement position opening 412′. Once all of the applicator tip engagement position openings have engaged the applicator tip and energy applied, the first template guide 410 may be removed from the base frame and the next template 411 may be inserted in the template window 404. In FIG. 4C, the second template guide 411 also includes projections 418, 418′ that are complimentary may engage with the index members 408 and 408′ of the base frame. In FIG. 4C, the template guide also includes five applicator tip engagement position openings 413, 413′, 413″, 413′″, 413′ that are all offset as compared with the relative positions of the applicator tip engagement position openings in the first template guide 410. Thus, the combination of the four (4) applicator tip engagement position openings in the first template guide and the five (5) applicator tip engagement position openings in the second template guide allow for the precise and accurate positioning of the applicator tip in each of the 9 subregions covering the full treatment area. In any of these methods the base frame may be removed and repositioned (or a new base frame and template guides may be repositioned) if additional treatment coverage is desired.

FIGS. 5A-5B illustrate examples of template guides within a base frame, similar to the variation shown in FIGS. 4A-4C. In FIG. 5A the base frame 502 is a rectangle with an index member 508 in the upper left corner that may match a cut-out region in the corner of the template guides. The template guide 511 in FIG. 5A is similar to the second template guide 411 in FIG. 4C having five (four peripheral and one central) applicator tip engagement position openings. The same base frame 502 is shown in FIG. 5B with a different template guide 510, which is similar to the first template guide 410 shown in FIG. 4B.

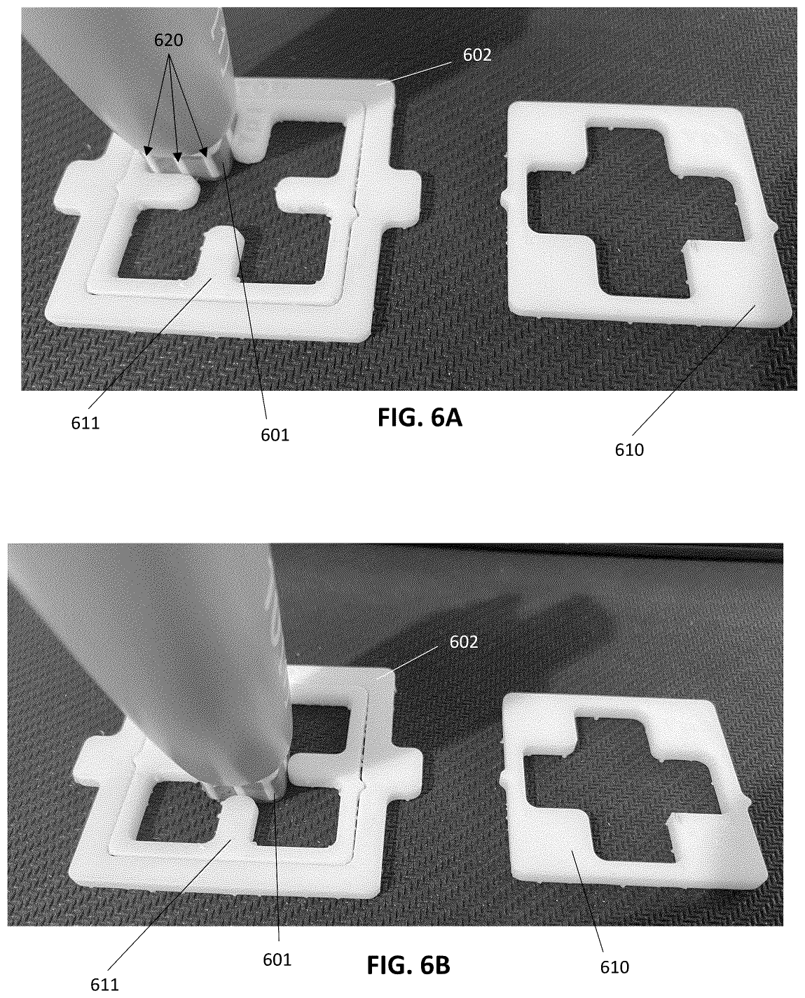

FIGS. 6A-6B illustrate the operation of an example template apparatus similar to that shown in FIGS. 4A-4C and 5A-5B. In FIG. 6A the template apparatus includes an outer base frame 602 holding a first inner template guide 611. A second template guide 610 having a non-overlapping pattern of applicator tip engagement position openings is shown to the right. In FIG. 6A an applicator tip 601 is shown engaged within a first applicator tip engagement position opening in the upper left of the template guide 611. In use the base frame may be secured (e.g., taped and/or attached with its own adhesive backing) to the skin. The tip applicator 601 may be used to apply energy from each of the five (5) applicator tip engagement position openings in the first template guide 611, while the tip applicator is held in a first orientation (e.g., with the three (3) line markings 620 pointing in the same direction). For example, FIG. 6B shows the tip applicator 601 inserted into a different applicator tip engagement position opening as compared to FIG. 6A. After the first template guide is removed from the template window of the base frame 602 and the second template guide is inserted into the base frame (indexed appropriately), the tip applicator may be rotated 90 degrees and energy may be applied from each of the four applicator tip engagement position openings in the second template guide 610 (not shown). As mentioned, in general the apparatus (e.g., frame, templates, etc.) may be provided with an adhesive, such as but not limited to, an adhesive tape, and/or the user may provide adhesive to a back surface of the apparatus (e.g., frame, templates, etc.).

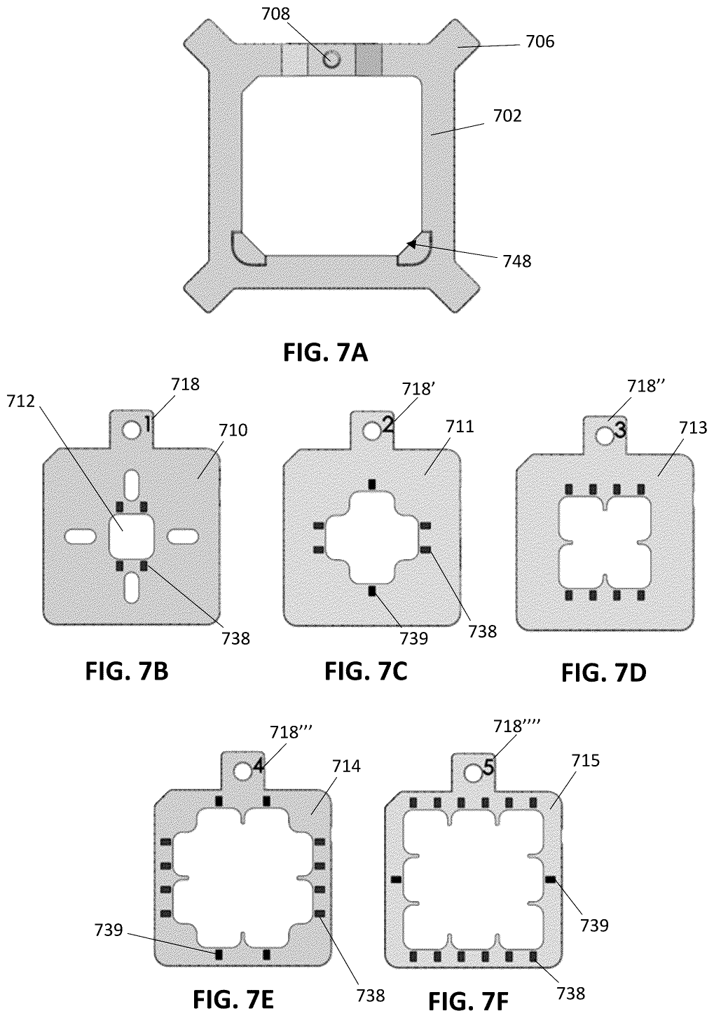

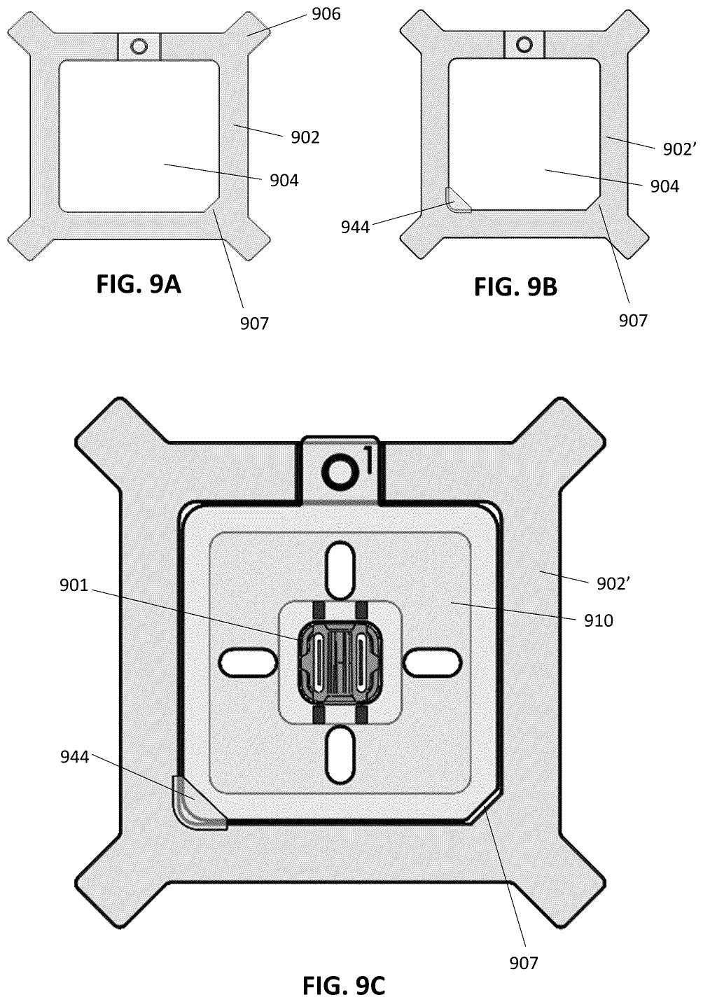

FIGS. 7A-7F illustrate another example of a template apparatus including a base frame having attachment projections 706 to which an attachment material (e.g., adhesive, bandage, cloth surgical tape, etc.) may be used to secure the frame 702 to the patient over the target tissue. The base frame also includes an index member 708 (shown here as a tab or post) that engages with the complimentary projection 718, 718′, 718″, 718′″, 718″″ on the template guides 710, 711, 713, 714, 715 shown in FIGS. 7B-7F. The example of the base frame shown in FIG. 7A also includes a second index member 748 in the bottom right corner, which is in this example in a form of a pocket that engages or holds the corner of the template guides when it is inserted into the base frame. The example of FIG. 7A has two pockets 748, however, in different implementations any number of pockets may be used, for example, 1, 2, 3 or 4 pockets. Further, in some examples instead of the pockets 748, one or more inner corners of the base frame may have a cut-out region that matches the corresponding cut-out region in the corner of the template guides for engagement with the base frame, as shown below, for example, in reference to FIG. 9A.

FIGS. 7B-7F show template guides (numbered 1-5 on the complimentary projections 718, 718′, 718″, 718′″, 718″). Each of the template guides 710, 711, 713, 714, 715 includes one or more applicator tip engagement position openings (a single applicator tip engagement position opening 712 is shown in FIG. 7B). In this example, each applicator tip engagement position opening is shown with a rotational orientation indicator that, in this example, comprises colored markings 738, 739 that are configured to match markings on the applicator tip to rotationally orient the applicator tip relative to the base frame and template guide (and therefore the tissue). In this example, either one line 739 or two lines 738 are shown, corresponding to the number of lines (one or two) on the adjacent sides of the applicator tip.

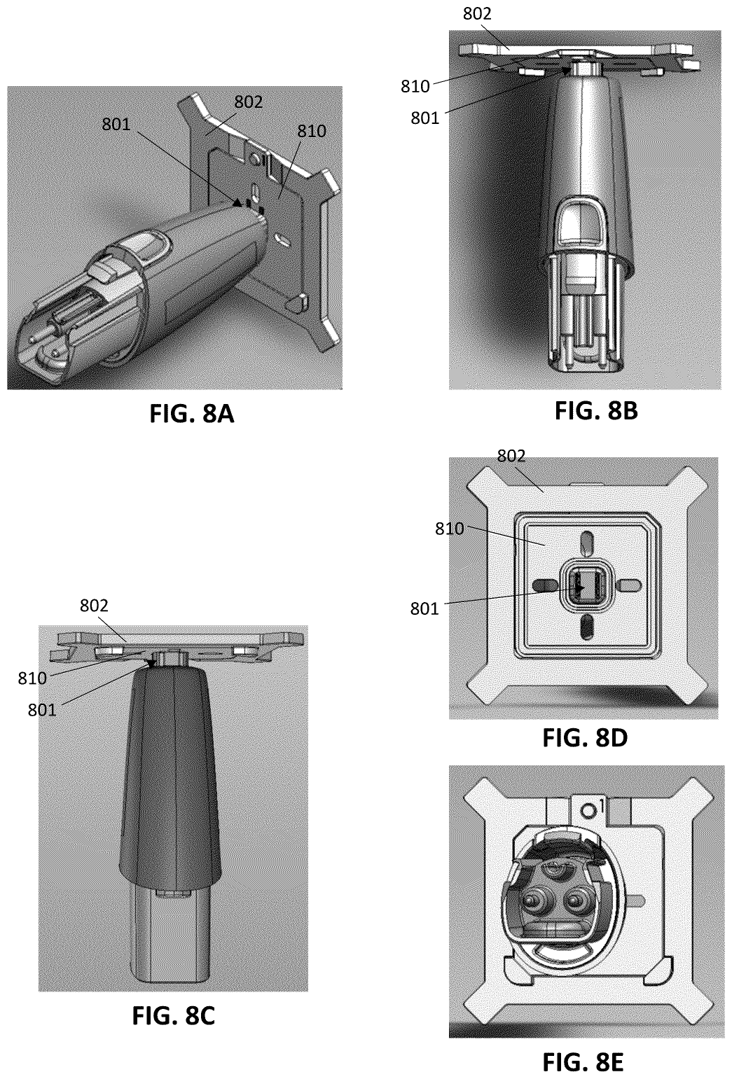

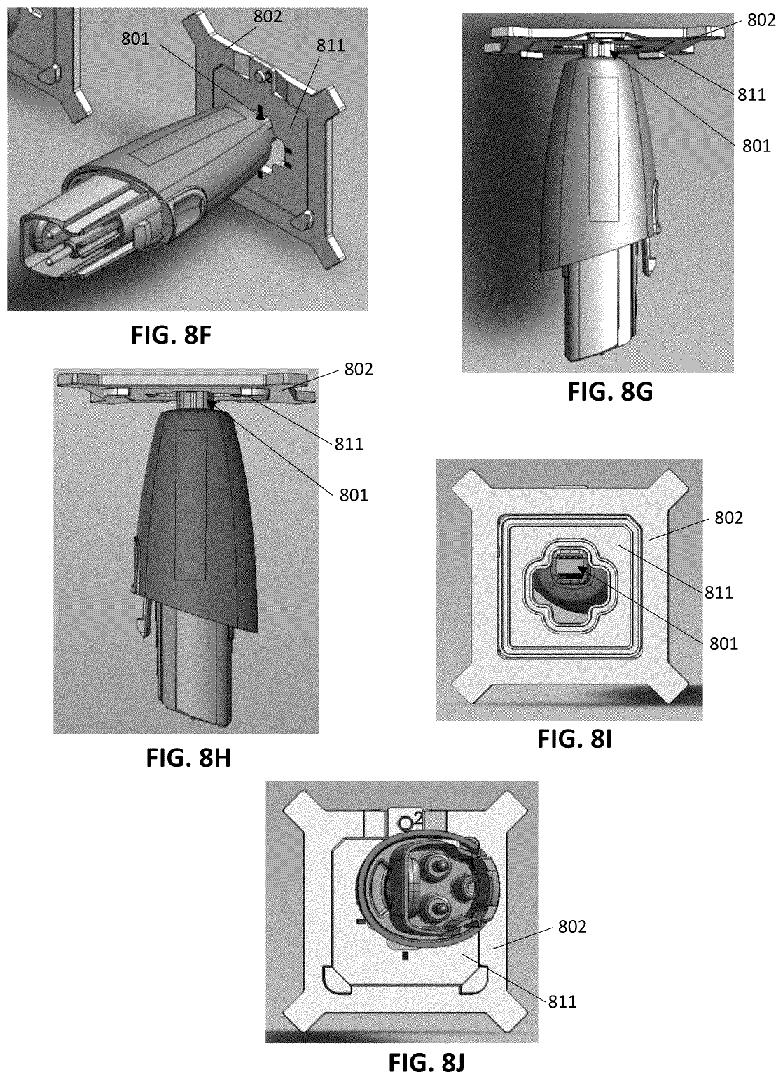

In FIG. 7A-7F, the template apparatus is configured to engage with the applicator tip 25 times, e.g., the total of five template guides includes a total of 25 applicator tip engagement position openings through which the applicator tip may be inserted. FIGS. 8A-8E illustrate an example of an applicator tip 801 being guided by the various applicator tip engagement position openings. FIG. 8A shows a top perspective view of the base frame 802 holding a first template guide 810 with the applicator tip 801 inserted into the single applicator tip engagement position opening. FIGS. 8B and 8C show back and front views. FIGS. 8D and 8E show bottom and top views, respectively. For comparison, FIGS. 8F-8J show a second template guide 811 within the base frame 802, and the same applicator tip 801 inserted in one of the four tip engagement position openings.

The base frames shown in FIGS. 4A-8J are square frames that include one or more index members to hold the template guides in position. Any appropriate base frame may be used, including base frames that are not square, but are rectangular, oval, round, polygonal (e.g., five sided, six sided, seven sided, eight sided, etc.). The base frame may include a lock or latch for releasably securing the template guide in position. FIGS. 9A and 9B illustrate examples of base frames. The base frame 902 of FIG. 9A is similar to that shown in FIGS. 7A and 8A-8J and has four attachment projections 906 and a window 904. Also, the base frame of the example of FIG. 9A has an engagement member in a form of a notch or cut-out 907 in the lower right corner of the base frame that mates with the corresponding notch in the template guide, which may be used to help to position and removably secure a template guide in the template window 904. In FIG. 9B the base frame 902′ in addition to the notch/cut-out 907 also includes an engagement member 944 that may be used to help removably secure a template guide in the template window 904. This is illustrated in FIG. 9C, showing an example of a template guide 910 secured within the template window of the base frame 902′. One corner of the template guide is indexed (e.g., notched so that it mates with the index member/engagement member 907 of the base frame), and the adjacent corner is secured by the engagement member 944. In this example a portion of an applicator tip 901 is shown inserted into the applicator tip engagement position of the template guide.

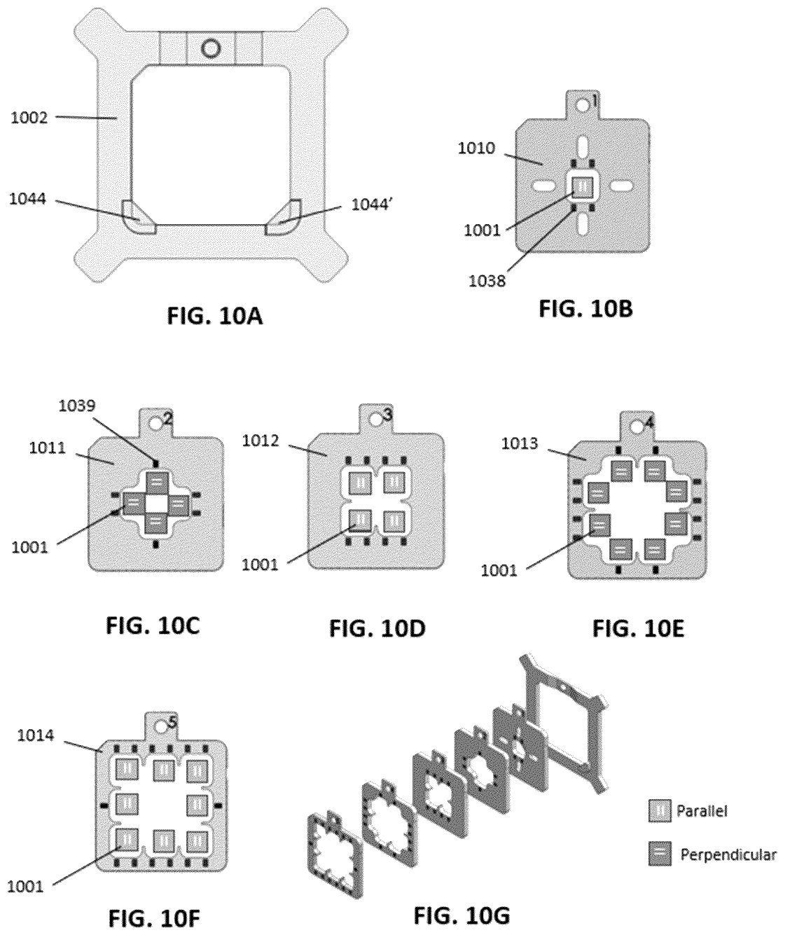

FIGS. 10A-10F illustrate another example of a template apparatus including a base frame 1002 with a pair of engagement members 1044, 1044′. FIGS. 10B-10F illustrate examples of five template guides that may be sequentially used with the base frame shown in FIG. 10A. in each of these examples a model of an applicator tip, showing the angular orientation of the applicator tip, e.g., parallel or perpendicular, relative to the long axis (e.g., up/down in FIGS. of the base frame. In each of the template guides shown 1010, 1011, 1012, 1013, 1014 the various applicator tip engagement position openings are all shown with a model applicator tip 1001 inserted. The model applicator tips are marked to show the rotational orientation of the electrodes in the tip. As can be seen, for each template guide the same applicator tip orientation may be used, as indicated by the rotational orientation indicators 1038, 1039. This arrangement may be particularly beneficial, as it may save time and may be much easier for the user to operate, without having to rotate the applicator tip. Thus, the template guides may be configured so that the final pattern includes a checkerboard pattern of alternating rotational orientations, but each template guide may include rotational orientation indicators that do not require the user to rotate the applicator tip within each template in order to achieve this checkerboard pattern. FIG. 10G illustrates a perspective view of the base frame (on the right side) and each template guide sequentially positioned to the left of the base frame.

As mentioned, FIGS. 10A-10G show examples of the change in orientation (e.g., parallel or perpendicular needle orientations) in which the tip may be rotated when being applied in some of the template openings. This change in orientation of the tips, and therefore the electrodes, may provide a superior treatment effect, including providing a cosmesis when healing (e.g., preventing or limiting scarring, etc.).

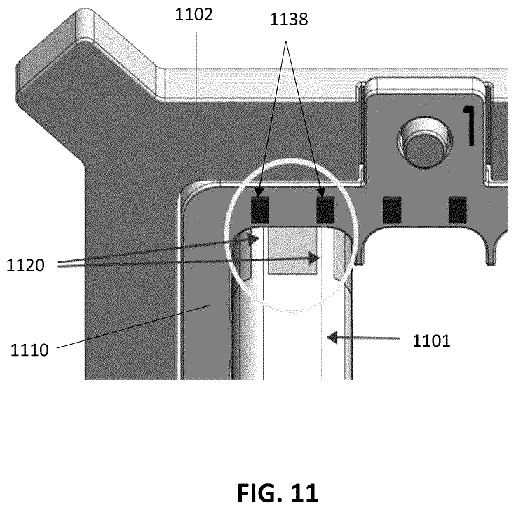

For example, FIG. 11 illustrates a rotational orientation indicator 1138 on a template guide 1110. The template guide 1110 is secured within the base frame 1102, and an applicator tip 1101 is engaged with a first applicator tip engagement position opening, so that rotational orientation indicators 1138 (shown as a pair of lines) are aligned with a pair of makers 1120 on the applicator tip, as shown. As mentioned, rotation of the tips between adjacent treatments may prevent deleterious effects due to the needle penetration and/or application of energy via the needle electrodes.

In general, the template apparatuses described herein may be used with any appropriately sized tip and may include margin regions between the electrode tips. The margin may refer to the region between the tip contact regions on the tissue (e.g., the gap or space between these contact regions. The margin may be positive (e.g., 1 mm margin, 2 mm margin, 3 mm margin, 4 mm margin, 5 mm margin, etc.) or negative; a negative margin means that the tip areas or tip contact regions overlap. The negative margin may be, e.g., −1 mm margin, −2 margin, −3 mm margin, etc.). In some examples the template may have at gap (positive margin) between the electrode tip contact regions because the effective treatment sizes may be larger than, for example, the nominal 5 mm×5 mm or 10 mm×10 mm area of the tip. Thus, the templates may be configured so that the positions of the applicator tip engagement openings are arranged so that there are not gaps of treatment. In some cases, the arrangement of the applicator tip engagement position openings may be selected based on the tissue being treated. For example, templates that may be useful for tissue that includes a high cellulose area may be configured with a negative margin (e.g., overlap) in order to prevent apparent gaps of treatment.

The template guides shown in FIG. 10A-10F are ordered sequentially (working their way out from the center of the pattern). The template guides may be used in the numbered order indicated (e.g., 1-5) or they may be used in any order (5 to 1, etc.).

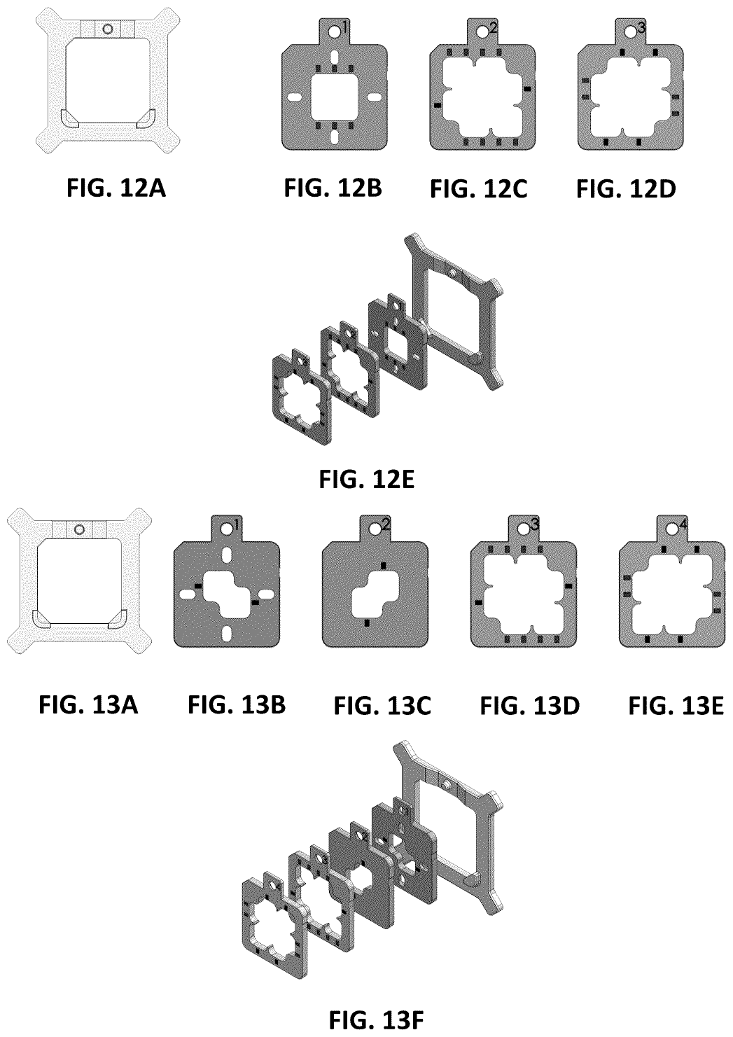

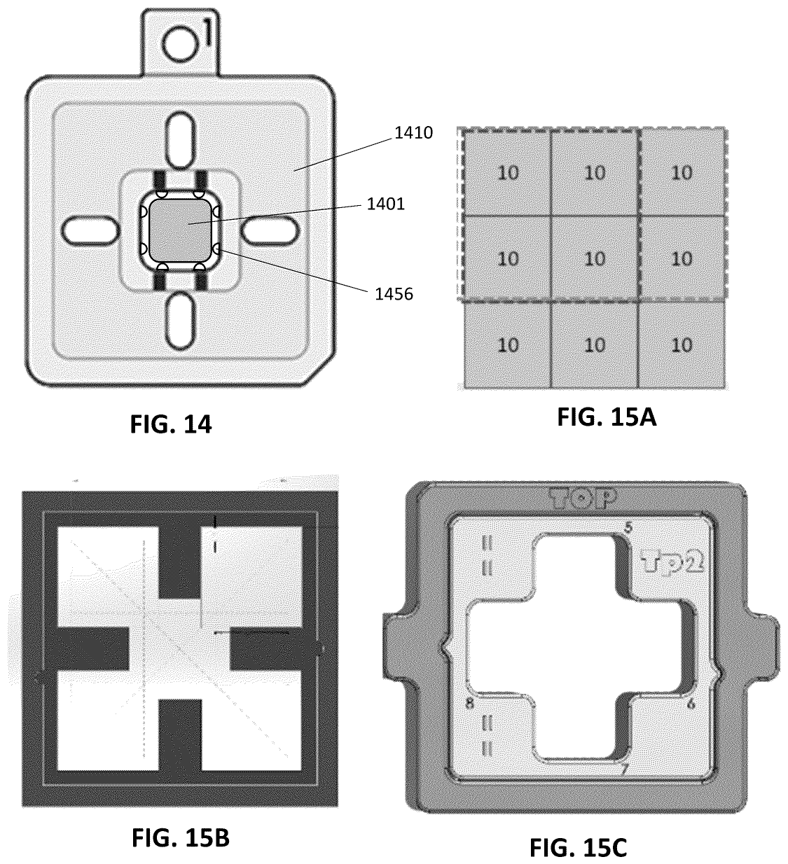

FIGS. 12A-12E and 13A-13F illustrate examples of template apparatus. In FIGS. 12A-12D the template apparatus includes a base frame (FIG. 12A) and three template guides (FIGS. 12B-12D). FIG. 12E illustrates a perspective view of the base frame (on the right side) and each template guide sequentially positioned to the left of the base frame. Two of the template guides (FIGS. 12C-12D) are similar to each other, but rotated 90 degrees; in some examples, only a single template guide is used, but may be configured to seat within the template window of the base frame in either of these orientations. In some examples, the template guides for both orientations are provided, and a user may use the appropriate template guide depending on the shape of the target tissue. In the non-limiting example shown in FIGS. 12A-12E, the treatment template is a 2 cm×2 cm treatment template with 12 applicator tip engagement position openings for a 5 mm×5 mm tip and one applicator tip engagement position opening for a 10 mm×10 mm tip.

In FIGS. 13A-13E the apparatus includes a base frame (FIG. 13A) and four template guides (FIGS. 13B-13E). FIG. 13F illustrates a perspective view of the base frame (on the right side) and each template guide sequentially positioned to the left of the base frame. FIGS. 13A-13F show a non-limiting example of a system including a 2 cm×2 cm treatment area having four templates with 16 applicator tip engagement position openings for a 5 mm×5 mm applicator tip. In some examples only two template guides may be used but may be used in two different rotational orientations within the base frame. In some examples, template guides for both orientations are provided, and a user may use the appropriate template guide depending on the shape of the target tissue.

In general, the applicator tip engagement position openings shown here are typically slightly larger than the size of the applicator tip so that the applicator tip may be easily inserted. However, in some cases it may be beneficial to limit the allowed movement (e.g., tolerance, gap) of the applicator tip within the applicator tip engagement position openings. In some examples the applicator tip engagement position openings may include one or more offsets (e.g., “tolerance offsets”) that may hold the applicator tip in position more securely. For example, FIG. 14 schematically illustrates an example of an apparatus with a plurality of protruding offsets 1456 around the inner perimeter of the applicator tip engagement position opening of the template guide 1410. An applicator tip 1401 is shown inserted into the applicator tip engagement position opening. In some examples the protruding offsets may be configured as bearings. The protruding offsets may be biased (e.g., coupled to a spring) to center the applicator tip within the applicator tip engagement position opening.

FIGS. 15A-15C illustrate another example of a template apparatus as described herein. In FIG. 15A an example of the pattern of the target treatment region is shown; the template guides shown in FIGS. 15B and 15C may be used to achieve the pattern of FIG. 15A. In the example shown in FIG. 15A, two alternative configuration of target treatment regions are shown, one having a 20 mm×20 mm treatment area and the other having a 20 mm×30 mm treatment area. In general, the sizes of the treatment area are not limited to blocks of 10 mm×10 mm, or to square areas. For example, as mentioned above, the applicator tip engagement position openings may be spaced, e.g., having positive or negative margin, and may be arranged offset from each other. Thus, although rectangular and square treatment patterns are illustrated herein, the methods and apparatuses, and in particular the treatment patterns, are not limited to this.

In any of these apparatuses the base frame and/or template guides may be formed of a material that conforms to the surface of the tissue (e.g., skin). The base frame and/or template guides may be formed of a relatively compliant material, such as but not limited to a polymeric material including a silicone, ethylene vinyl acetate (EVA), polyethylene (PE), thermoplastic elastomer (TPE), etc. having relatively softer durometers. In any of the apparatuses described herein the base frame and/or templates may be formed of a material having a durometer of between about 15 A and 60 A on the Shore A scale (e.g., 15A-50A, 35A-45A, etc.).

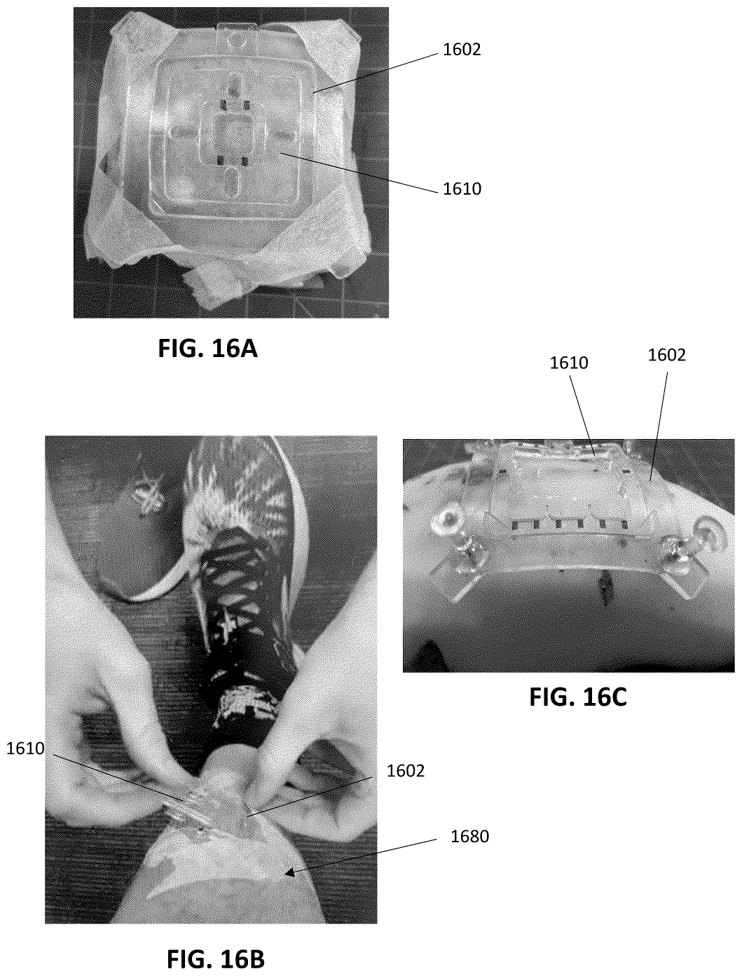

For example, FIGS. 16A-16C illustrate an example of a base frame 1602 that substantially conforms to the curved surface of the skin (e.g., leg 1680) and shows a template guide 1610 that also substantially conforms. In any of these apparatuses the frame may be positioned over the region to be treated and may be positioned over a gel material (including a nonconductive or conductive gel) to assist in the treatment. Any of the apparatuses described herein may include an engagement member or retainer, such a pocket or rib that may engage with the template, as described above. The flexible frame may also include one or more (e.g., 2, 3, 4) engagement members on the corner(s). For example in FIG. 16C the flexible frame includes four engagement members (e.g., ribs, pockets, etc.).

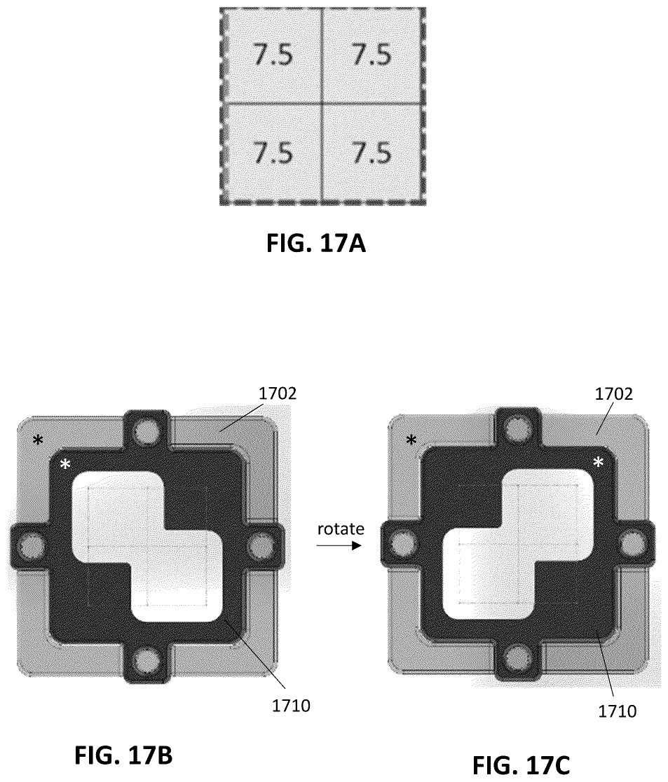

FIGS. 17A-17C also illustrate examples of template apparatuses as described herein. FIG. 17A shows an example of a pattern, divided up into subregions; the template guides shown in FIGS. 17B and 17C may be used to achieve the pattern of FIG. 17A. In this example, the same template guide 1710 may be used in the same base frame 1702 but rotated 90 degrees, as shown by the comparison between FIGS. 17B and 17C.

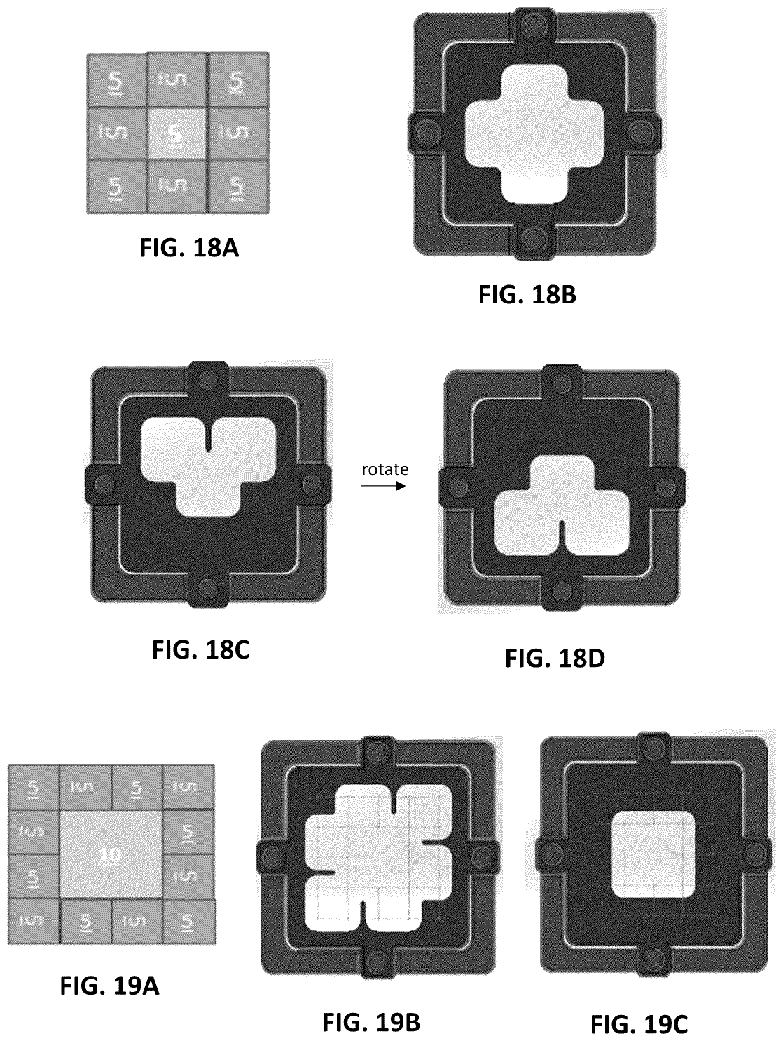

FIGS. 18A-18D and 19A-19C also illustrate examples of template apparatuses as described herein. FIG. 18A shows an example of a pattern for applying electrical therapy to a tissue, while FIGS. 18B-18D show the template guides that may achieve this pattern. The same template guide may be used (rotated 180 degrees) in FIGS. 18C and 18D. FIG. 19A shows a pattern for an electrical treatment, and FIGS. 19B and 19C show examples of template guides that may be used to achieve this pattern; the template guide in FIG. 19B may be rotated and positioned within the frame to achieve the pattern of FIG. 19A.

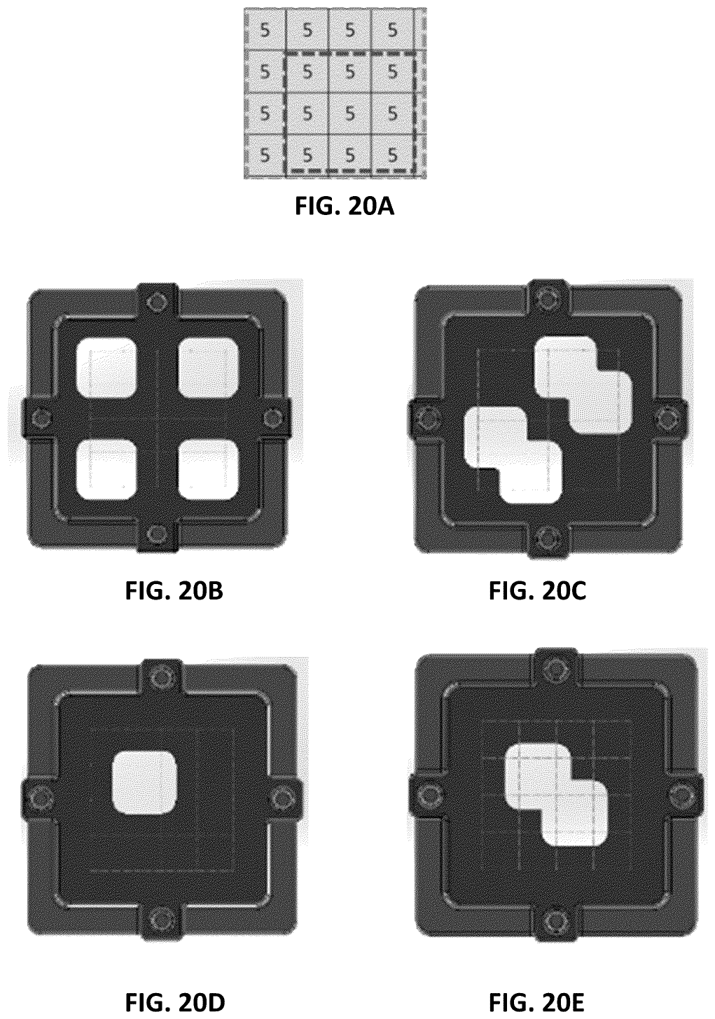

Similarly, FIGS. 20A-20E illustrate another example of a pattern (FIG. 20A) and a series of alternative template guides (FIGS. 20B-20E) that may be used to achieve this pattern. In this example a first set of template guides (e.g., the templates schematically shown in FIGS. 20B, and 20D) may be used or a second set of equivalent template guides (e.g., the templates schematically shown in FIGS. 20B, 20C and 20E) may be used to cover the same treatment area of FIG. 20A. If the first set is used, the first template (FIG. 20B) may be used once, the second template (FIG. 20C) may be used twice (in 2 orientations that are 90 degrees offset) and the third template (FIG. 20D) may be used four times in each of four orientations that are 90 degrees offset from each other to cover the center portion of the pattern. Alternatively, the second set of templates may also use the first template (FIG. 20B) once and the second template (FIG. 20C) twice, with a 90 degree rotation between each, while the third template (FIG. 20E) is used twice, with a 90 degree rotation between each use to achieve full coverage of the pattern of FIG. 20A.

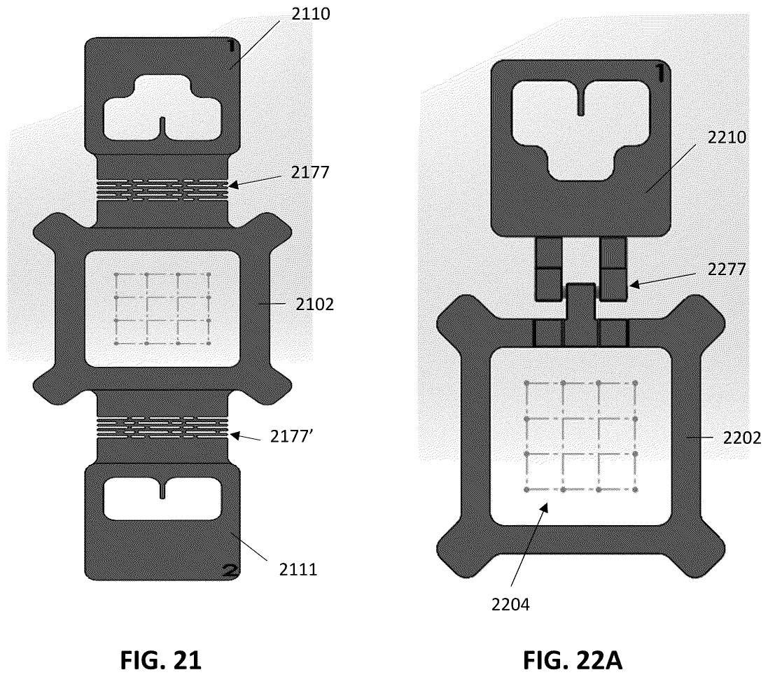

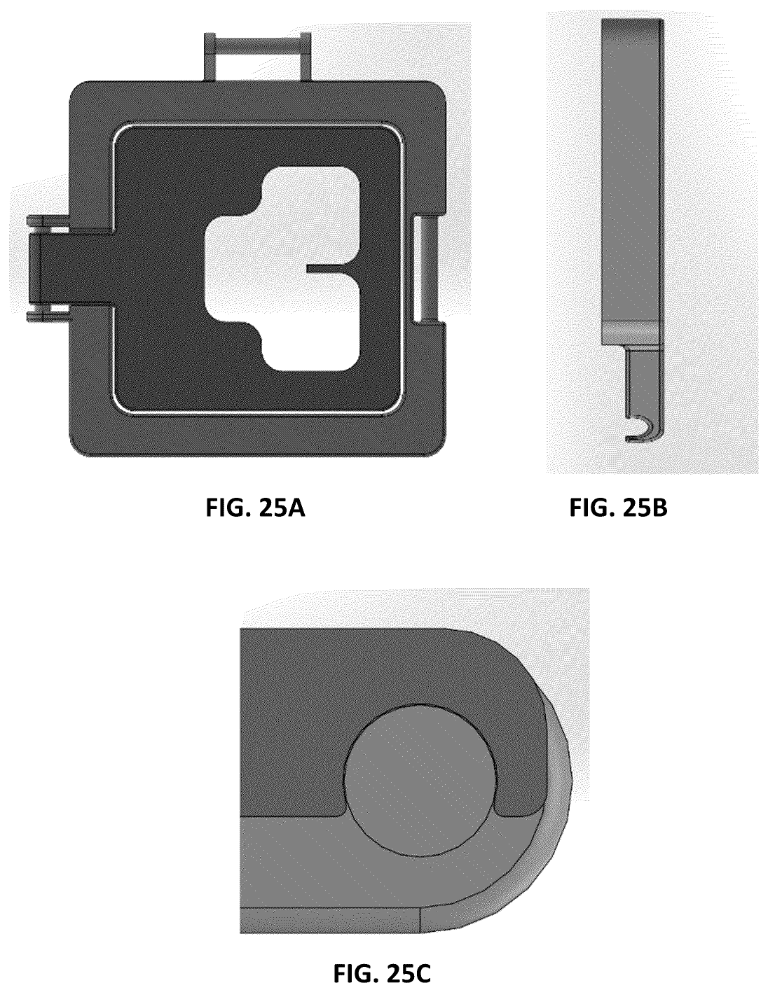

In any of the apparatuses described herein the base frame and the one or more template guides may be coupled together. For example, the template guide may be movably connected to the base frame. FIGS. 21-28 illustrate examples of this configuration. In FIG. 21 two template guides 2110, 2111 are movably coupled to the base frame 2102 via a pair of hinge regions 2177, 2177′. The hinge region may be a living hinge. In some examples the hinge region is formed by a stacked up kerf region. Although FIG. 21 shows only two template guides attached at a top and bottom, in some examples three or four template guides may be attached on the top, bottom, left and/or right sides of the base frame.

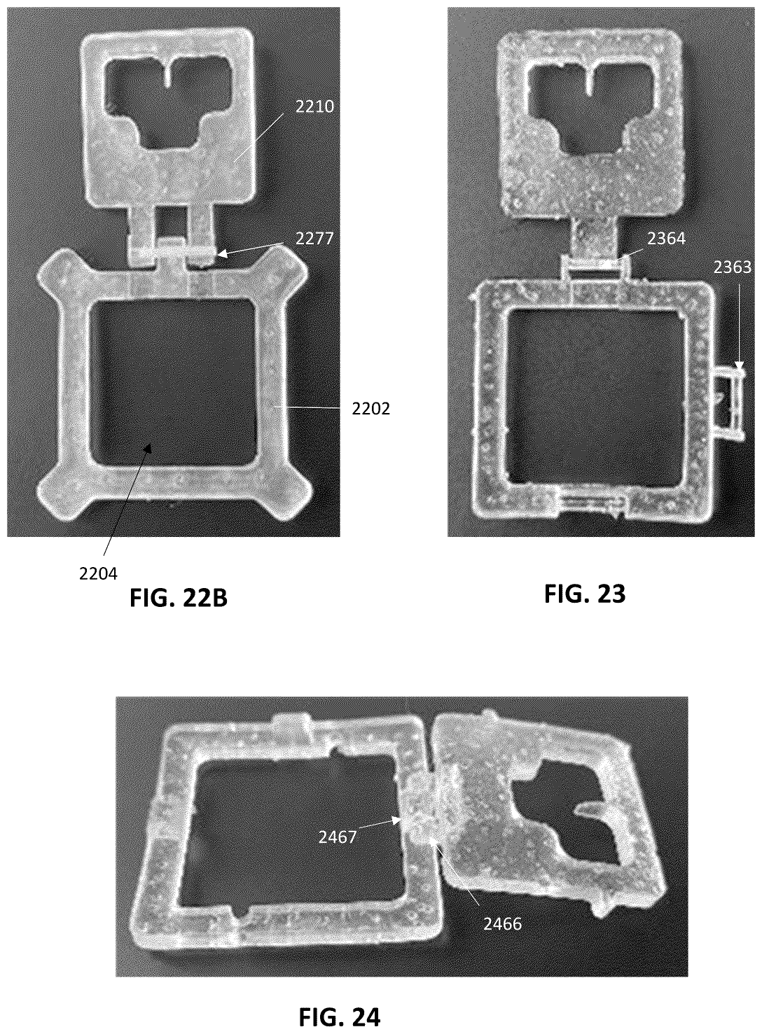

FIG. 22A-22B shows another example of an apparatus including an attached or attachable template guide that is hinged to the base frame, in this example by a dowel 2277 securing the two together so that they may rotate, e.g., so that the template guide 2210 may flip or fold over into the template window opening 2204 of the base frame 2202.

FIG. 23 shows a similar example of a prototype in which the template guides may be snap-fit onto a c-shaped rotating attachment (e.g., C-bracket), as shown in greater detail in FIGS. The base frame may include a bar region 2363 onto which the c-shaped pivoting member 2364 (that may connect to the template guide) is attached. The various template guides may then be snapped onto the bar region on one of the sides of the base frame. Other examples of attachments (including “snap attachments”) may include, for example, dowel and snap attachments.

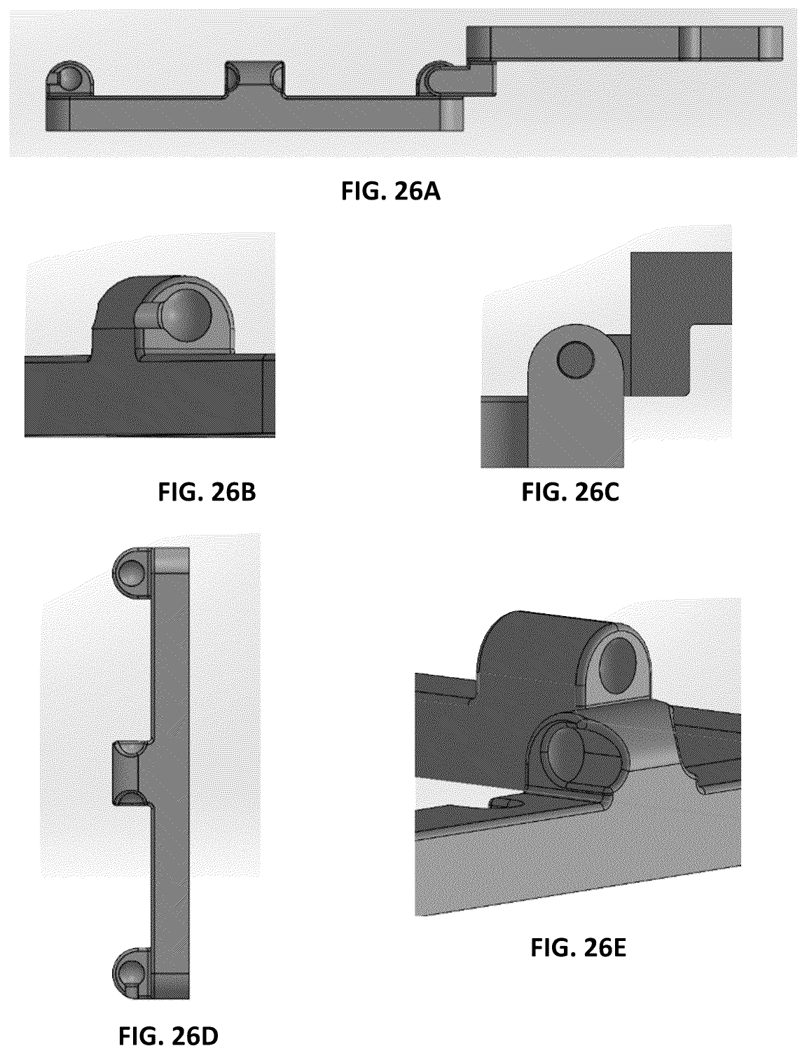

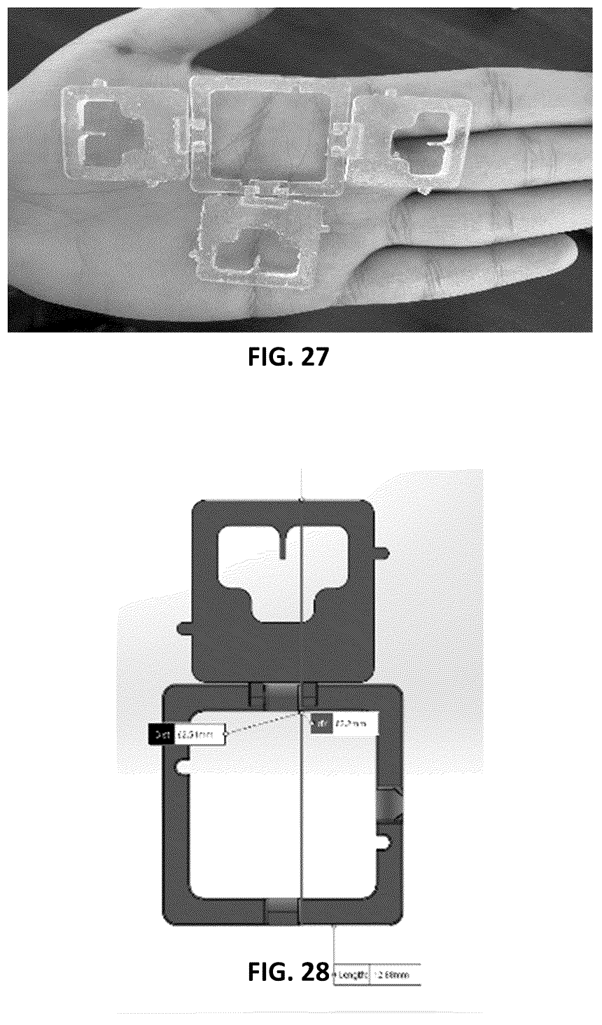

FIGS. 24 and 26A-26E illustrate a hinge region that is formed as a ball-and-socket configuration including a pair of ball regions 2466 that engage in tension with a socket region 2467, allowing them to pivot relative to each other. For example, FIG. 27 shows one example of an apparatus including a base frame and three hinged (via ball-and-socket hinged regions) template guides that may be sequentially inserted (and removed) from the template window. FIG. 28 shows another example of a hinged apparatus.

In any of the hinged configurations shown in FIGS. 22-28 (and in particular in the snap-in/snap-on configurations) more than four templates may be used, and may fit into the base, particularly when full coverage of a larger area is desired.

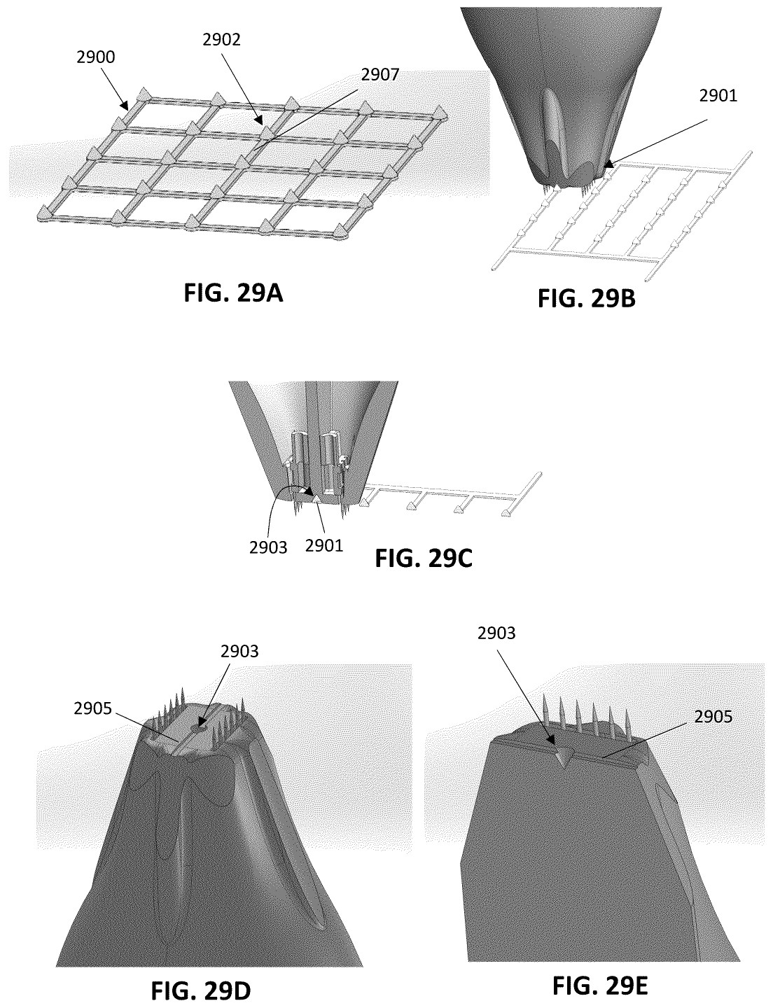

FIGS. 29A-29E illustrate another example of an apparatus. In this example, the template apparatus includes a grid (grid frame) having engagement structures/engagement members formed on the grid to guide the placement of the treatment tip (and therefore, electrodes). The grid may be referred to as a template guide, grid template guide, grid frame, or grid frame template guide. As in the templates guides described above, the grid is configured to receive and engage with a distal region of an applicator tip. Although FIGS. 29A-29E shows only a single grid, multiple grids may be used. In addition, the grid may be configured for use with a base frame as described above; e.g., a plurality of different grids (grid template guides) may engage with a base frame in one or more orientations to form a pattern on the tissue.

As shown in FIG. 29A, the grid 2900 may include tracks or channels having mating engagements (e.g., cones or pylons) 2902 into which the applicator tip(s) may engage so that the electrodes are properly aligned to apply electric treatment. In FIGS. 29B-29C the applicator tip 2901 is shown engaged with a mating engagement (e.g., cone) 2902 of the grid 2900 of FIG. 29A, and electrodes from the applicator tip may be extended into the tissue. The complimentary mating engagement 2903 of the applicator tip may be seated with the mating engagement 2902 of the grid. FIG. 29C shows a section through a distal end of the applicator tip 2901, showing a complimentary mating engagement 2903 (in this example, a cone-shaped cut-out or hole) in the applicator tip mating with the corresponding mating engagement 2902 of the grid. FIGS. 29D and 29E illustrate the applicator tip, including the complimentary mating engagement 2903. In some examples the mating engagement on the grid and/or the complimentary mating engagement of the applicator tip may be keyed or otherwise configured to rotationally orient the applicator tip relative to the tissue. Alternatively or additionally, as shown in FIGS. 29D and 29E, the applicator tip may also include a secondary mating engagement region, shown as a channel 2905 in FIGS. 29D and 29E, that engage with the frame 2900, such as with the struts 2907 forming the frame. Alternatively, in some examples the mating engagement of the grid may be configured to engage with an edge of the tip, so that the perimeter of the applicator tip may act as a complimentary mating engagement.

FIG. 29E shows a cross-section of the distal end of the applicator tip 2901 demonstrating a complimentary mating engagement configured as a cut-out (or a hole) 2903 having a shape corresponding to (and mating with) the mating engagement (e.g., cone in this example) 2902 on the grid, for engaging with the grid of FIG. 29A.

As mentioned, in some examples the grid 2900 could be configured and adapted to fit inside of a base frame similar to the base frames described in various examples of the present disclosure. Different grids, having alternative locations of the mating engagements (e.g., cones, etc.) may be provided and configured to fit within the base frame. Alternatively or additionally, the grid(s) may have an adhesive backing to keep them from moving/slipping. The adhesive may be on a back surface of the grid, including on a portion of the back or on the entire back surface of the grid. The adhesive may be a biocompatible adhesive (e.g., a skin-compatible adhesive). In some examples, the grids may have a peel-away surface to assist in accurate placement.

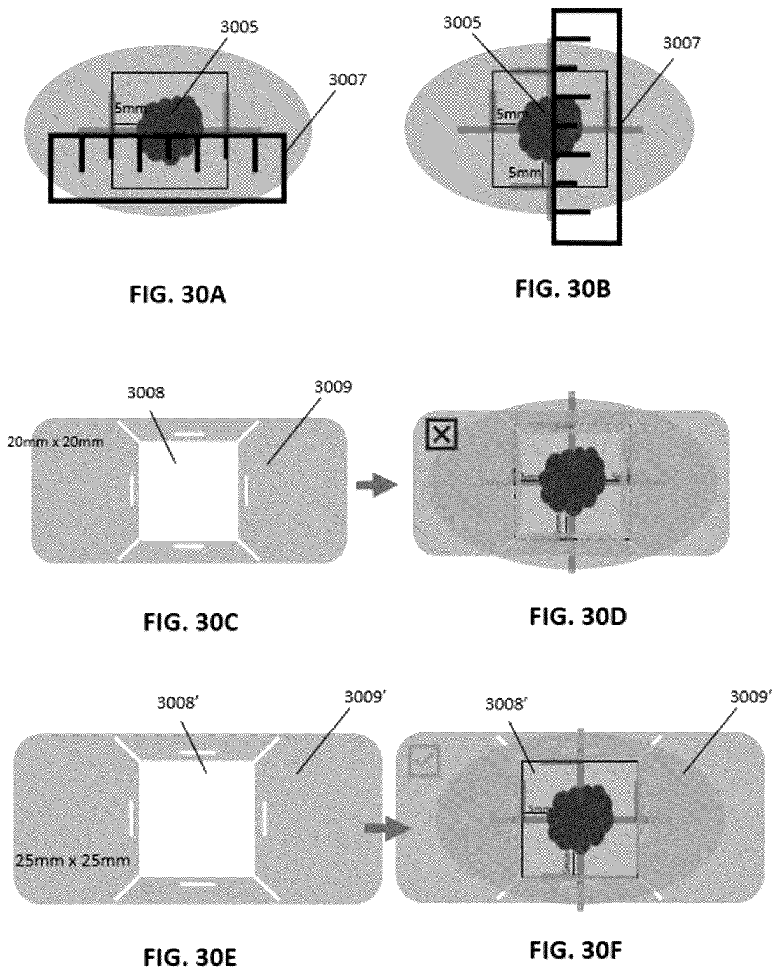

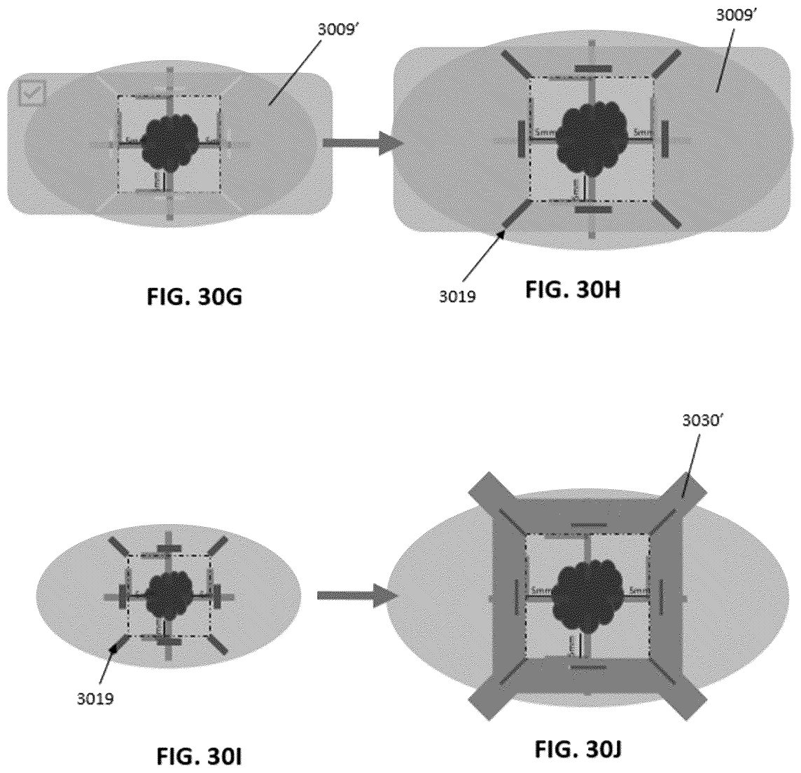

Also described herein are methods of selecting and placing the templates of the present disclosure relative to the tissue to be treated. In general, the dimensions of the treatment area may be determined and matched to a template to allow accurate treatment. For example, FIGS. 30A-30J illustrate one example of a method of selecting and placing a template (e.g., a base frame and a plurality of template guides) for treating a tissue lesion 3005. In FIGS. 30A and 30B the treatment area may be marked as shown, using a ruler 3007, and a margin spacing distance from the edge of the lesion may be identified (e.g., selected). In this example, the margin spacing distance is 5 mm all around the edges of the lesion forming a square. The ruler may be placed through the midpoint region of the lesions and is used to plot the edge of and mark the treatment area. Next, as shown in FIGS. 30C and 30D or alternatively FIGS. 30E and 30F, the appropriate template size may be determined by lining up the window 3008, 3008′ of a base pattern overlay 3009, 3009′ over the marked treatment area, as shown in FIG. 30D or 30F. In FIG. 30C the base pattern overlay 3009 is a 20 mm×20 mm window 3008 overlay, while in FIG. 30E the slightly larger base pattern overlay 3009′ is a 25 mm×25 mm window 3008′ overlay. In this example, the 20 mm×20 mm of the smaller overly 3009 does not fit well, and the marks are not within the window 3008. However, the overlay having the 25 mm×25 mm window in FIG. 30F is sufficiently large, and the marks are all within the open window 3008′.

Once the appropriate base pattern overlay has been determined, base alignment markings 3019 may be applied from the overlay 3009′ onto the tissue (e.g., skin), as shown in FIGS. 30G-30H. In FIG. 30H the alignment markings 3019 have been made through the overlay 3009′. Thereafter, these alignment markings may be used to apply and align a base frame 3030, as shown in FIG. 30J, and the base frame may be secured (e.g., adhesively secured) to the tissue, as described above.

It should be appreciated that all combinations of the concepts and features discussed (provided such concepts and features are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed. Further various features shown in different examples may be combined, or one or more features from one example may be implemented in a different example.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A template apparatus for guiding an application of electrical energy to a tissue, the apparatus comprising:
a base frame; and
one or more template guides configured to removably fit into the base frame in one or more orientations,
wherein each template guide of the one or more template guides includes one or more applicator tip engagement position openings configured to receive a distal region of an applicator tip, and
further wherein the one or more applicator tip engagement position openings of the one or more template guides are configured to form an enlarged treatment pattern after at least one of the one or more template guides held within the base frame in more than one orientation and/or more than one of the one or more template guides are sequentially held within the base frame so that the applicator tip may be received by the one or more applicator tip engagement position openings of the one or more template guides.

2. The template apparatus of claim 1, wherein the base frame comprises an adhesive configured to secure the base frame to a treatment site.

3. The template apparatus of claim 1, further comprising attachment projections extending from the base frame and configured to attach the base frame to the tissue.

4. The template apparatus of claim 1, further wherein at least some of the applicator tip engagement position openings include one or more rotational orientation indicators configured to rotationally align the applicator tip within the applicator tip engagement position openings.

5. The template apparatus of claim 4, wherein the one or more rotational orientation indicators comprise markings configured to match markings on the applicator tip.

6. The template apparatus of claim 4, wherein the one or more rotational orientation indicators comprise projections configured to engage with the applicator tip in a particular orientation.

7. The template apparatus of claim 1, further comprising an indexing member on the base frame and configured to engage each template guide of the plurality of template guides and to releasably orient the template guide in the base frame.

8. The template apparatus of claim 7, wherein the indexing member comprises a tab configured to engage with a tab receiver on each template guide of the plurality of template guides.

9. The template apparatus of claim 1, wherein the plurality of template guides is movably attached to the base frame.

10. The template apparatus of claim 9, wherein the plurality of template guides is hinged to the base frame by one or more of: a dowel pin, a c-shaped, a ball joint, and a living hinge.

11. The template apparatus of claim 1, further comprising an engagement member on each base frame configured to engage with each template guide to removably secure the template guide in the base frame and against the tissue.

12. The template apparatus of claim 11, wherein the engagement member comprises a pocket, a tab or a latch.

13. The template apparatus of claim 1, wherein the base frame is flexible and configured to conform to a curved tissue surface.

14. The template apparatus of claim 1, wherein the enlarged treatment pattern formed by the applicator tip engagement position openings of the plurality of template guides is configured to provide continuous and non-overlapping coverage of the tissue.

15. The template apparatus of claim 1, wherein at least some of the plurality of template guides are configured to removably fit into the base frame in two or more rotational orientations that are offset from each other.

16. The template apparatus of claim 15, wherein the two or more rotational orientations are offset from each other by 90 degrees or 180 degrees.

17. A template apparatus for guiding an application of electrical energy to a tissue, the apparatus comprising:
a base frame; and
a plurality of template guides configured to removably fit into the base frame in one or more orientations, wherein each template guide of the plurality of template guides includes one or more applicator tip engagement position openings configured to receive a distal region of an applicator tip, and
further wherein the one or more applicator tip engagement position openings of a first template guide of the plurality of template guides forms an enlarged treatment pattern with: 1) the one or more applicator tip engagement position openings of a second template guide of the plurality of template guides, or 2) the one or more applicator tip engagement position openings of the first template guide in a second orientation of the one or more orientations.

18. The template apparatus of claim 17, wherein the second orientation is rotated by 90 degrees, 180 degrees, and/or 270 degrees relative to a first orientation.

19. The template apparatus of claim 17, wherein the second template guide of the plurality of template guides is configured to fit into the base frame such that locations of the applicator tip engagement position openings of the second template guide do not coincide with respective locations of the applicator tip engagement position openings of the first template guide.

20. A template apparatus for guiding the application of electrical energy to a tissue, the apparatus comprising:
a base frame;
a plurality of template guides configured to removably fit into the base frame in a predetermined orientation, wherein each template guide of the plurality of template guides includes one or more applicator tip engagement position openings configured to receive a distal region of an applicator tip and to hold the applicator tip in a fixed position and orientation relative to the tissue, further wherein a least some of the applicator tip engagement position openings include a rotational orientation indicator configured to align with and/or engage a complementary indicator on the distal region of the applicator tip.
